(19) United States
(12) Patent Application Publication
(10) Pub. No.: US 2025/0281393 A1
(43) Pub. Date: Sep. 11, 2025

(54) BODY CAVITY GEL

(71) Applicant: CS MEDICA A/S, Copenhagen (DK)

(72) Inventor: Lone HENRIKSEN, Fredensborg (DK)

(21) Appl. No.: 18/564,550

(22) PCT Filed: May 25, 2022
(86) PCT No.: PCT/EP2022/064271

§ 371 Date: Nov. 27, 2023

(30) Foreign Application Priority Data
May 25, 2021 (DK) PA202170268

(51) Int. Cl. A61K 9/00; A61K 9/06; A61K 31/00; A61K 47/02; A61K 47/10; A61K 47/14; A61K 47/18; A61K 47/22; A61K 47/24; A61K 47/26; A61K 47/36; A61P 31/16

(52) U.S. Cl. CPC A61K 9/0043; A61K 9/06; A61K 31/658; A61K 47/02; A61K 47/10; A61K 47/14; A61K 47/18; A61K 47/22; A61K 47/24; A61K 47/26; A61K 47/36; A61P 31/16

(57) ABSTRACT

The present invention relates to non-psychoactive cannabinoid-comprising compositions, said compositions comprising cannabidiol (CBD) formulated for application in a body cavity, such as a gel for intranasal application. Said composition comprises CBD; glycol(s); emulsifier(s), including non-ionic emulsifier(s); NaCl; panthenol; hyaluronic acid/salt; phytic acid and water, and optionally one or more of gelling agent, allantoin, and/or pH regulator. Such compositions can be used in the context of protection against pathogens and/or in reduction of ingress of infectious and/or irritating agents such as dust, pollen, microorganism(s), bacteria, fungi, and/or virus, such as COVID-19.

### FIELD OF THE INVENTION

The present invention relates to a non-psychoactive cannabinoid-comprising composition, such as a composition comprising cannabidiol (CBD) formulated for application in a body cavity, such as a gel for intranasal application. Such composition can be used in the context of protection against pathogens and/or in reduction of ingress of infectious and/or irritating agents such as dust, pollen, microorganism(s), bacteria, fungi, and/or virus, such as COVID-19.

### BACKGROUND OF THE INVENTION

WO2021056109 “Gel base composition for compounding into a mucoadhesive delivery system” relates to mucoadhesive concentrated base compositions having high viscosity of at least 50,000 cPs, which can be processed upon dilution into a gel dosage form and upon drying into a strip dosage form for use as a vehicle for delivery of active ingredients to mucocutaneous surfaces, such as the oral, rectal, nasal or vaginal cavities.

US20200000693 “Compositions Comprising Silicon Dioxide-Based Particles Including One or More Agents” concerns compositions that can be applied to a mucous membrane. In contrast, inter alia, the present invention does not comprise SiO2-based particles.

US20100273895 “Formulations of Cannabidiol and Prodrugs of Cannabidiol and Methods of Using the Same” concerns body cavity-administrable gel compositions. However, in contrast to the present invention, e.g. several components of the compositions and/or their concentrations are not disclosed.

US20120202891 “Cannabinoid-Containing Compositions and Methods for their Use” pertains to predominantly orally administered cannabinoid gel formulations.

WO2020024056 “Compositions Comprising Cannabinoids and Absorbable Material and uses thereof” concerns a composition which can be administered inter-nasally. However, in contrast to the present invention, e.g. several components of the compositions and/or their concentrations are not disclosed.

US20200170962 “Nasal cannabidiol compositions” concerns a pharmaceutical gel composition comprising cannabidiol. However, in contrast to the present invention, e.g. several components of the compositions and/or their concentrations are not disclosed.

Seasonal influenza is a common acute respiratory tract infection that leads to about 291 243-645 832 respiratory deaths globally each year]. Currently, strains from 2 sub-types of influenza A (H3N2, H1N1) and 2 lineages of influenza B vira (Yamagata, Vic-toria) are the major causes of seasonal epidemics. However, whether the severity of the illness caused by these influenza vira is clinically similar in adults is controversial. For example, epidemiologic studies indicate that influenza A (H3N2) subtype infections have caused higher influenza-associated hospitalizations and mortality among seasonal vira, whereas recent hospital-based studies have suggested that clinical outcomes such as length of stay, mortality, pneumonia, hospitalization, intensive care unit (ICU) ad-mission, and death did not differ by virus type. A systematic analysis pointed out that most studies have been based on population influenza surveillance or limited numbers of clinical cases and concluded that little evidence existed to show differences in the severity of illness caused by seasonal influenza vira. Therefore, more comprehensive studies are required to evaluate the comparative severity of illness caused by the 2 vira in those hospitalized. During the 2017-2018 season, the percentage of clinical labora-tory-tested specimens positive for the B/Yamagata lineage increased markedly in China, largely consistent with findings from the US Centers for Disease Control and Prevention. Consequently, we conducted a prospective observational study to compare the clinical features and outcomes between hospitalized patients with laboratory-con-firmed influenza A and B virus infection.

Concerning the COVID-19 pandemic, in Mark W. Tenforde et al. (2021) “Identifying COVID-19 risk through observational studies to inform control measures”, JAMA. 2021; 325 (14): 1464-1465, the authors conclude the importance of wearing masks and the clustering of transmission have been shown with COVID-19, with 20% of infected individuals estimated to cause about 80% of SARS-COV-2 transmissions. Clear evidence supports the effectiveness of simple strategies in identifying risks and mitigating the spread of infection, with much of this evidence coming from observational studies.

There is a need for formulations, such as intra-nasal formulations, for preventing, reducing and/or mitigating the risk of infection, allergies, or other symptoms that can be triggered by e.g. air-born particles, such as dust, pollen, microorganism(s), bacteria, fungi, and/or viruses, such as COVID-19. It is thus an object of the present invention to provide formulations that can be applied in a body cavity, such as intra-nasally, that fulfil this need.

### SUMMARY OF THE INVENTION

In a first aspect, the present invention concerns a composition suitable for and/or formulated for application in a body cavity, said composition comprising cannabidiol (CBD), glycol(s), non-ionic emulsifier(s), NaCl, panthenol, hyaluronic acid/salt, phytic acid and water. In some embodiments, the composition may further comprise one or more of: gelling agent, allantoin, and/or pH regulator. In some embodiments, the composition is formulated as a gel, such as an intra-nasal gel. In some embodiments, such a composition or gel is formulated with a neutral or slightly acidic pH, such as a pH around 5-7. In some embodiments, the composition comprises (by weight) one or more of: (a) cannabidiol (CBD): 0.05-2.5%; (b) glycol(s): 12-70%; (c) non-ionic emulsi-fier(s): 0.5-5.0%, (d) NaCl: 0.5-2.5%; (e) panthenol: 0.25-2.0%; (f) hyaluronic acid/salt: 0.2-2.5%; (g) phytic acid: 0.05-1.0%; (h) water: up to 100%; (i) gelling agent: 0.5-5.0%; (j) allantoin: 0.015-1.5%; and/or (k) pH regulator: 0.02-1.0%; including any combination(s) thereof.

In a second aspect, the present invention pertains to a method for providing a composition according to the first aspect. In some embodiments, provision of a composition according to the present invention comprises the steps or acts of:
Providing the individual components in suitable amounts
Providing of first composition by mixing water; glucol(s) such as etylene glycol and butylene glycol, an emulgator, such as a non-ionic emulgator, e.g. Eumulgin VL 75; NacCl; panthenol; hyaluronic acid/hyaluronate; and optionally allantoin, such as by stirring, until completely dissolved;
Adding Phytic acid;
Adjusting pH (optional), such as by addition of NaOH;
Providing a second composition by dissolving CBD in propylene glycol;
Combining the first and the second composition;
Stirring the combined compositions until homogeneous; and optionally
Dispensing the composition in receptacles, such as receptacles according to the sixth aspect.

Optionally, in particular when formulating a composition as a gel, such a method will comprise the act of:
Adding a gelling agent, such as hydroxyethylcellulose, under mixing, e.g. after combining the first and the second composition.

In a third aspect, the present invention relates to a composition provided by a method according to the second aspect.

In a fourth aspect, the present invention concerns a composition according to the first or third aspect for use as a medicament and/or in the treatment, prophylactic treatment, mitigation and/or alleviation of one or more symptom(s) and/or condition(s) related to infectious agents, such as dust, pollen, environmental pollution, bacteria and/or vira, including any combination(s) thereof. In some embodiments, the composition provides e.g. one or more of:
prevention of allergy by particles, such as pollen, dust, hair and the like;
mitigation and/or reducing the severity of allergy, such as by pollen, dust, hair and the like;
prevention of infection by pathogens, such as vira, microorganisms, bacteria, yeasts or fungi;
mitigation of risk of infection by airborne pathogens, such as vira, microorganisms, bacteria, yeasts or fungi; and/or reducing severity of an infection by pathogens, such as vira, microorganisms, bacteria, yeasts or fungi.

In a fifth aspect, the present invention concerns a method of treatment, prophylactic treatment, mitigation and/or alleviation of one or more symptom(s) and/or condition(s) comprising the use of a composition according to the first, third or fourth aspect, wherein the one or more symptom(s) and/or condition(s) is/are related to infectious agents, such as dust, pollen, environmental pollution, bacteria and/or vira, including any combination(s) thereof.

In a sixth aspect, the present invention relates to a receptacle comprising a composition according to first, third or fourth aspect.

In a seventh aspect, the present invention concerns a kit comprising a receptacle according to the sixth aspect, and optionally a packaging.

In an eighth aspect, the present invention concerns a CBD-comprising composition, such as a topical composition, e.g. a composition suitable and/or formulated for application in a body cavity, such as a nasal gel, wherein the CBD used in the formulation is crystalline. In some embodiments, the CBD is of “type A” (needle-like crystals) or capable of forming needle-like crystals.

In a ninth aspect, the present invention pertains to a dosage regimen, comprising administering a topical composition, in particular CBD-comprising topical composition as disclosed herein. In some embodiments, the CBD is of “type A”.

### DESCRIPTION OF THE DRAWINGS/FIGURES

FIG. 1: microscope picture of cannabinol (CBD) forming needle-like crystals. The CBD crystals were sourced from www.enecta.com.

FIG. 2: microscope picture of cannabinol (CBD) forming cluster- or bunch-like crystals. The CBD crystals were sourced from www.pharma-hemp.com.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Thus, the term “comprising” is generally to be interpreted as specifying the presence of the stated parts, steps, features, or components, but does not exclude the presence of one or more additional parts, steps, features, or components. For example, a composition comprising a chemical compound may thus comprise additional chemical compounds.

Generally, compositions as disclosed herein, in particular topical compositions such as body cavity compositions may comprise one or more pharmaceutically acceptable ad-juvant(s), such as pharmaceutically acceptable carrier(s), excipient(s), stabilizer(s), salt(s) and/or buffer(s) or the like.

Where used herein, terms like “for example”, “e.g.” or “such as”, particularly when followed by a listing of terms, is merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition. Thus, unless indicated otherwise, “%” indicates “% weight/weight (w/w)”, also called “weight %” or “% by weight”.

In the context of the present invention, the terms “about”, “around”, “approximately” or the symbol “˜” can be used interchangeably, and are meant to comprise variations and/or uncertainties generally accepted in the field, e.g. comprising analytical errors and the like. Thus “about” may also indicate measuring uncertainty commonly experienced in the art, which can be in the order of magnitude of e.g. +/−1, 2, 5, 10, or even 20 percent (%). Furthermore, “about” may be understood to refer to numbers in a range of numerals, for example the range of +/−20, +/−15, +/−10, +/−5, +/−2, +/−1, +/−0.5, +/−0.1% of the referenced number. Moreover, all numerical ranges herein should be understood to include all integers, whole or fractions, within the range.

In the context of the present invention, a “drop” or “droplet” can be used interchangeably. As used herein, a drop can e.g. be a volume of around 1/20 of a ml and/or g. A drop can also be larger or smaller, such as in the range of 0.01-1.0, 0.015-0.1, 0.025-0.075, or around 0.05 g. In some embodiments, the volume of one drop is around 10-250, 15-100, 25-75, or ˜50 μl.

As used herein, the term “in some embodiments” is meant to comprise “in one embodiment”, “in some embodiments”, and “in one or more embodiments”.

In the context of the present invention, the terms “subject” or “patient” can be used interchangeably, and are meant to comprise a human, animal and/or mammal. In particular, a human subject can e.g. be selected from one or more of: female, male, senior, adult, adolescent, child, or infant. An animal subject can e.g. be selected from pet, hus-bandry, mammal, reptile, bird, and/or animal in a zoo. A patient can also be a “potential patient”, i.e. a healthy subject at risk to get infected and/or exposed to undesired compounds, such as dust, pollen, environmental pollution, bacteria and/or vira, including any combination(s) thereof.

In the context of the present invention, the term “treatment” is meant as an act aiming at preventing, mitigating, alleviating, lessen, improving and/or curing any symptom(s), condition(s), or disease(s) in a subject. In the context of the present invention, the term “treatment” may also comprise a prophylactic treatment. The effect of the treatment may also comprise reduction in pain and/or discomfort. A treatment may also result in a faster recovery and/or healing compared to a control. A further effect of a treatment may also comprise a recovery/healing with less complications compared to a control. A control can e.g. be no treatment or treatment with a placebo. Generally, a “treatment” in the present context comprises topical application of a suitable amount of a body cavity composition into said body cavity, once or several times per day, as e.g. further elucidated herein.

Without wanting to be bound by any theory, it is believed that use of a body cavity composition as disclosed herein may result in increased tolerance to undesirable compounds, such as dust, pollen, environmental pollution, bacteria and/or vira, including any combination(s) thereof. This tolerance can e.g. be the need for a higher dose of such undesirable compounds for triggering a reaction, such as infection and/or allergy.

In some embodiments, use of the body cavity composition may reduce the risk of con-taminating other subjects, such as healthy subjects, with an infectious disease, such as common cold, or influenza, including COVID-19.

“Skin” can be described as the layer of usually soft, flexible outer tissue covering the body of an animal, in particular vertebrate animal. The three main functions of the skin are believed to be protection, regulation, and sensation. The skin is believed to comprise three layers, the (i) epidermis, (ii) dermis and (iii) hypodermis, also called subcutaneous tissue.

A “mucous membrane” or “mucosa” is a membrane that lines various cavities in the body and covers the surface of internal organs. It consists of one or more layers of epi-thelial cells overlying a layer of loose connective tissue. It is mostly of endodermal origin and is continuous with the skin at different body openings such as the eyes, ears, inside the nose, inside the mouth, lip, vagina, the urethral opening, prepuce, and the anus. Some mucous membranes secrete mucus, a thick protective fluid. The function of the mucous membrane is to stop pathogens and dirt from entering the body and to prevent bodily tissues from becoming dehydrated. Mucous membranes can be contiguous with skin, such as at the nostrils, the lips of the mouth, the eyelids, the ears, the genital area, and the anus. Along with providing a physical barrier, they also contain key parts of the immune system and serve as the interface between the body proper and the microbiome.

A “body cavity” in the context of the present invention can e.g. be selected from: eyes, ears, inside the nose (also called “nostril” herein), inside the mouth, lip, vagina, urethral opening, prepuce and the anus. A nostril can also be defined as one of the two channels of the nose, from the point where they bifurcate to the external opening.

In some embodiments, the body cavity composition is formulated for intra-nasal application, i.e. be applied onto the skin/mucous membrane in the nostril(s) of a subject, usually the within the last 1 or 2 cm of the channel close to the external opening. Usually, this distance correlates roughly to the length of the external opening to the beginning of the nose bone and/or the bony part of the nasal septum of an adult human. In some embodiments, this distance is also called along the lines of “slightly inside/into the nostril”. Concerning dosage, a common dosage comprises application of one drop of e.g. around 50 mg or μl pr. nostril of an average sized nose/nostril of an adult subject. Further dosages are disclosed herein.

In a first aspect, the present invention concerns a body cavity composition comprising CBD, and at least one penetrator(s) and/or penetration enhancer(s). Generally, the composition is formulated for topical application in a body cavity, such as a mucous-tissue-comprising body cavity, e.g. a nostril. In some embodiments, the composition can be formulated as a gel, and/or for intra-nasal application. A composition formulated for e.g. intra-nasal application formulated as a gel can be called “nose gel” herein.

In some embodiments, such a body cavity composition may comprise:
a) 0.1-5%, 0.12-2%, 0.15-1%, 0.18-0.75%, or around 0.2% (w/w) cannabidiol (CBD);
b) 25-85%, 35-75%, 45-70% or 50-65, or around 53% (w/w) water; and
c) one or more penetrator(s) and/or penetration enhancer(s) such as propylene glycol and/or pentylene glycol.

Cannabidiol, CAS no. 3956-29-1 is a non-psychoactive cannabinoid. It can be provided in different purities, and is usually extracted from Cannabis sativa by methods known in the art. In the context of the present invention, CBD with a high degree of purity is generally preferred, such as “crystalline” CBD, comprising neither oil nor further cannabinoids, such as psychoactive or non-psychoactive cannabinoids in significant amounts.

In particular, when absence of oil(s) and/or fat(s) is desired, common sources of CBD, such as CBD-comprising oils are not desirable. Thus, in some embodiment, CBD is provided in essentially pure form, such as in crystalline or powder form and/or with a purity of 95%, 98%, 99%, 99.5%, 99.8% or more than 99.8%. Without wanting to be bound by any theory, it is believed that the use of CBD in crystalline may further contribute in a positive fashion, such as that less CBD is required to provide a similar effect compared to a crude CBD preparation. This is surprising, as according to general belief, further cannabinoids present in such crude CBD preparations are believed to provide a synergistic effect.

Generally, the water used in the formulations is of drinking water quality. It may also be distilled or deionized water, such as “MilliQ water”.

CBD-comprising compositions described herein usually comprise one or more skin penetrating enhancer(s), such as a mixture of two or more skin penetration enhancers. Commonly, a “skin penetrating enhancer”, “penetrating enhancer” or “penetrator”-all three terms can be used interchangeably herein-improves the ability of one or more relevant component(s)/ingredient(s) of the composition, such as CBD to pass through the epidermal layer and the dermal layer of the skin to reach the adipose tissue that underlies the skin wherein adipocytes are increased in number and/or size. Without wanting to be bound by any theory, it is believed that this may be accomplished by a number of different mechanisms including, for example, by extracting lipids from the stratum corneum, increasing the partitioning of the active ingredients into the skin, and disrupting the lipid bilayer of the stratum corneum, thus rendering the stratum corneum structure more fluid and increasing the ability of the composition including the canna-binoids to diffuse through the stratum corneum.

In some embodiments, the “penetrator enhancer” is provided in a concentration of 0.1-15% (w/w), 0.5-12% (w/w), 1.0-10% (w/w), 2.0-7.5% (w/w), 4.0-6.0% (w/w), or around 5% (w/w). Suitable skin penetrating enhancers can be, for example, sulfoxides, alcohols, fatty acids, fatty acid esters, polyols, amides, surfactants, terpenes, alkanones, and organic acids, among others. Specific examples of suitable sulfoxides include di-methylsulfoxide (DMSO) and decylmethylsulfoxide, among others. Suitable alcohols include alkanols such as ethanol, propanol, butanol, pentanol, hexanol, octanol, n-octanol, nonanol, decanol, 2-butanol, 2-pentanol, and benzyl alcohol; fatty alcohols, such as caprylic alcohol, decyl alcohol, lauryl alcohol, 2-lauryl alcohol, myristyl alcohol, cetyl alcohol, stearyl alcohol, oleyl alcohol, linoleyl alcohol, and linolenyl alcohol; and isopropyl alcohol. Examples of suitable fatty acids include linear fatty acids such as valeric acid, heptanoic acid, pelagonic acid, caproic acid, capric acid, lauric acid, myristic acid, stearic acid, oleic acid, and caprylic acid; and branched fatty acids, such as isovaleric acid, neopentanoic acid, neoheptanoic acid, neononanoic acid, trimethyl hexanoic acid, neodecanoic add, and isostearic acid. Examples of suitable fatty acid esters include aliphatic fatty acid esters such as isopropyl n-butyrate, isopropyl n-hexanoate, isopropyl n-decanoate, isopropyl myristate, isopropyl palmitate, and octyldodecyl myristate; alkyl fatty acid esters such as ethyl acetate, butyl acetate, methyl acetate, methylvalerate, methylpropionate, diethyl sebacate, and ethyl oleate; and diisopropyl adipate and dimethyl isosorbide. Examples of suitable polyols include propylene glycol, butylene glycol, polyethylene glycol, ethylene glycol, diethylene glycol, triethylene glycol, dipropylene glycol, ethoxydiglycol, pentylene glycol, glycerol, propane-diol, butanediol, pentanediol, hexanetriol, and glycerin. Examples of suitable amides include urea, dimethylacetamide, diethyltoluamide, dimethylformamide (DMF), dimethyloctamide, dimethyldecamide, biodegradable cyclic urea (e.g., 1-alkyl-4-imidazo-line-2-one), pyrrolidone derivatives, biodegradable pyrrolidone derivatives (e.g., fatty acid esters of N-(2-hydroxyethyl)-2-pyrrolidone), cyclic amides, hexamethylene-lauramide and its derivatives, diethanolamine, and triethanolamine. Examples of pyrrolidone derivatives include 1-methyl-2-pyrrolidone, 2-pyrrolidone, 1-lauryl-2-pyrrolidone, 1-methyl-4-carboxy-2-pyrrolidone, 1-hexyl-4-carboxy-2-pyrrolidone, 1-lau-ryl-4-carboxy-2-pyrrolidone, 1-methyl-4-methoxycarbonyl-2-pyrrolidone, 1-hexyl-4-methoxycarbonyl-2-pyrrolidone, 1-lauryl-4-methoxycarbonyl-2-pyrrolidone, N-cyclo-hexylpyrrolidone, N-dimethylaminopropylpyrrolidone, N-cocoalkypyrrolidone, N-tal-lowalkylpyrrolidone, and N-methylpyrrolidone. Examples of cyclic amides include 1-dodecylazacycloheptane-2-one (e.g., Azone), 1-geranylazacycloheptan-2-one, 1-famesylazacycloheptan-2-one, H3,7-dimethyloctyl) azacycloheptan-2-one, 1-(3,7, 11-trimethyldodecyl) aza-cyclohaptane-2-one, 1-geranylazacyclohexane-2-one, 1-geranylazacyclopentan-2,5-dione, and 1-famesylazacyclopentan-2-one.

In some embodiments, the penetrator/penetration enhancer is or comprises a polyol. In some embodiments, the penetrator(s), such as a polyol, in particular a glycol is/are present in a concentration of 2-60%, 10-55%, 30-50% or around 40% (w/w). In some embodiments, the penetrator/penetration enhancer is or comprises a glycol. In some embodiments, the penetrator/penetration enhancer is or comprises propylene glycol. In some embodiments, the penetrator/penetration enhancer is or comprises pentylene glycol. In some embodiments, the penetrator/penetration enhancer is or comprises butylene glycol. In some embodiments, the penetrator/penetration enhancer is or comprises at least two of propylene-, butylene- and pentylene glycol. In some embodiments, the one or more penetrator(s) and/or penetration enhancer(s) is or comprises propylene glycol, butylene glycol and/or pentylene glycol. In some embodiments, the penetrators comprise at least two of propylene glycol, butylene glycol, and pentylene glycol, and a further glycol. In some embodiments, the composition comprises around 20-40%, or around 30% by weight propylene glycol. In some embodiments, the composition comprises around 2-10%, or around 5% butylene glycol. In some embodiments, the composition comprises around 2-10%, or around 5% pentylene glycol. In some embodiments, the composition comprises at least two of: around 20-40%, or ˜30% by weight propylene glycol; around 2-10%, or ˜5% butylene glycol; and around 2-10%, or ˜5% butylene glycol.

In some embodiments, CBD can be dissolved in the penetrator, such as propylene glycol. This advantage can e.g. be exploited in the production process. Commonly, CBD is dissolved in oil or alcohol. However, in some embodiments, oil and/or alcohol can be undesired, e.g. for the reasons discussed herein.

A body cavity composition as disclosed herein may also comprise one or more pharmaceutically acceptable adjuvant(s). In some embodiments, the adjuvant can be selected from one or more of: antioxidant(s), emulsifier(s), pH regulating agent(s), such as acid(s), base(s) or salt(s) thereof, stabilizer(s), colorant(s), including any combinations thereof.

Often, the body cavity composition will comprise one or more further agents, such as one or more gelator(s), one or more emollient(s), one or more skin conditioner(s), one or more wound healing compound(s) (e.g. hyaluronic acid and/or CBD), one or more anti-microbial agent(s), one or more pH stabilizer(s); one or more chelating agent(s), and/or NaCl, including any combination(s) thereof.

In some embodiments, the composition may thus comprise optionally one or more of (i)-(vii), such as:
i. One or more gelator(s), such as polyacrylic acid/polyacrylate, e.g. 2-propeonic acid, homopolymer, Carbomer) and/or hydroxyethyl cellulose;
ii. One or more skin moisturizer(s) and/or skin conditioner(s), such as panthenol and/or allantoin;
iii. One or more further wound healing compound(s), such as hyaluronic acid and/or its salt;
iv. One or more anti-microbial agent(s), such as benzalkonium chloride;
V. One or more pH stabilizer(s) and/or buffering agent(s); such as aminomethyl propanol (AMP);
vi. One or more chelating agent(s), such as phytic acid and/or its salt; and/or
vii. NaCl, such as 0.9% (w/w) and/or in a physiological concentration.
   including any combination(s) of (i)-(vii).

The body cavity composition can be formulated for topical application, such as a gel. A body cavity composition formulated as a gel provides e.g. the advantage of facilitating appropriate dosage and application, as well as one or more further advantages, as disclosed herein.

Thus, in some embodiments, the composition comprises one or more gelator(s) to provide a gel-like composition. A “gelator” or “gelling agent” is a substance or compound capable of forming a gel. Gels can e.g. be hydrogels, comprising a polymer or colloidal network. Examples of suitable gelators may comprise: 1-methyl-2,4-bis(N′-n-octade-cylureido) benzene (MBB18), 1-methyl-2,4-bis(N′-n-dodecylureido) benzene (MBB12), bis(4′-stearamido phenyl) methane (BSM18), bis(4′-octanamido phenyl) methane (BOM8), 12-hydroxystearic acid, nucleobase, phenylalanine, d-glucosamine, RAD 16, EAK 16, RAD 16 I, RAD 16-II, KLD-12, Nucleopeptide (phenylalanine di-peptide link to a nucleobase), Guanosine derivatives, Carbomer, such as Carbomer 910, 934, 940, 941 and 934P (these numbers are an indication of molecular weight and the specific components of the polymer), IKVAV-Peptide amphiphiles, Heparin-binding peptide amphiphile LRKKLGKA-PA, A glycosylated amino acetate type of hydrogela-tor 1 C33012N3H55, Gelator 4b (a derivative of d-gluconolactone) C1607N2H24, Unimer U-15, Unimer U-151, Unimer U-1946, and/or Unimer U-6. In some embodiments, the gelator can be selected from one or more gelators and/or gelling agent(s) disclosed herein.

In some embodiments, the one or more gelator(s) is provided in a concentration of 0.1-5%, 0.2-3% (w/w), 0.5-2% (w/w), 0.6-1.0% (w/w), or around 0.8% (w/w). In some embodiments, the gelator(s) is or comprises Carbomer, polyacrylic acid/polyacrylate, such as sodium polyacrylate, and/or hydroxyethyl cellulose. In some embodiments, the gelator is or comprises polyacrylic acid, polyacrylate, and/or 2-propeonic acid homopolymer. In some embodiments, the gelator is or comprises Carbomer. Poly(acrylic acid) (PAA; trade name Carbomer) is a synthetic high-molecular weight polymer of acrylic acid. The IUPAC name is poly(1-carboxyethylene). They may be homopoly-mers of acrylic acid, or crosslinked with an allyl ether of pentaerythritol, allyl ether of sucrose, or allyl ether of propylene. In a water solution at neutral pH, PAA is an anionic polymer, i.e. many of the side chains of PAA will lose their protons and acquire a neg-ative charge. This makes PAAs polyelectrolytes, with the ability to absorb and retain water and swell to many times their original volume. In some embodiments, the gelator is hydroxyethyl cellulose, such as 1-2.5%, or around 1.75% by weight. In some embodiments, the sole gelator and/or gelling agent is hydroxyethyl cellulose, such as 1-2.5%, or around 1.75% by weight. In some embodiments, the sole gelator and/or gelling agent is hydroxyethyl cellulose, such as 1-2.5%, or around 1.75% by weight, and the NaCl concentration is around 0.9% by weight.

In some embodiments, hyaluronic acid/hyaluronate acts as a gelator, either alone, or in combination with a further gelator and/or gelling agent. In some embodiments, the gelator is polyacrylic acid/polyacrylate and/or the gelling agent is hydroxyethyl cellulose. Hyaluronic acid is a polymer of disaccharides, which are composed of D-glucu-ronic acid and N-acetyl-D-glucosamine, linked via alternating β-(1->4) and β-(1->3) glycosidic bonds. Hyaluronic acid can be 25,000 disaccharide repeats in length. Polymers of hyaluronic acid can range in size from 5.000 to 20.000.000 Da in vivo. The average molecular weight in human synovial fluid is 3-4 million Da, and hyaluronic acid purified from human umbilical cord is 3.140.000 Da; other sources mention average molecular weight of 7 million Da for synovial fluid. Hyaluronic acid combines with water and swells to form a gel. Furthermore, hyaluronic acid is believed to be involved in tissue regeneration and is used as a dermal filler for e.g. facial wrinkles, as hyaluronic acid is known to bind and absorb water up to 1000 times its own molecule weight. In some embodiments, hyaluronic acid/hyaluronate acts as gelator, in combination with a further gelator, such as Carbomer or the like.

The function of the gelator can be described as the provision of a gel or gel-like texture of the composition. Thereto, one or more thickeners or gelling agents can be provided. Such thickeners/gelling agent(s) can be selected from acrylate cross polymers, in particular C10-C30 alkyl acrylate cross polymers (such as commonly marketed under the tradename Carbopol®), hydroxyethyl cellulose, xanthan gum and/or any combinations thereof. The amount of gelator and/or thickener(s) can be considered sufficient when it ensures that the gel does not run off during application. In some embodiments, the gel may comprise a thickener and/or gelling agent selected from acrylate cross polymers, hydroxyethyl cellulose, xanthan gum and/or any combinations thereof.

In some embodiments, the gelling agent or gelator is or comprises hydroxyethyl cellulose, such as Tylose H 300 NG4. Such a gelling agent can e.g. act as a binder and/or thickening agent. It is available in granular form with non-delayed solubility, which can be advantageous.

In some embodiments, the composition comprises one or more skin moisturizer(s) and/or one or more skin conditioner(s).

Generally, the terms “moisturizer”, “skin moisturizer”, or “emollient” can be used interchangeably and are meant to comprise a cosmetic composition providing protection, moisturizing and/or lubrication of the skin. A moisturizer may also prevent dryness and irritation of the skin by moisturization. In the context of the present invention, the term “moisturizer” or “emollient” may also relate to the individual compound(s) that provide or improve such a moisturizing effect. Examples of such compounds may comprise: Panthenol, Allantoin, Isopropyl Myristate, pantothenic acid, Sodium Hyaluronate, Squalene, Phenoxyethanol, methyl-paraben, propyl-paraben, ethyl-paraben, butyl-paraben, lanolin, sorbitol, petrolatum, Stearic acid, Shea butter, Glyceryl stearate, Elastin, Hyaluronic acid, Olive oil, Glycerine Pharma 99.5% vegetable gum, rhizobian, and/or Sea Water.

In some embodiments, the emollient is or comprises panthenol. In some embodiments, the emollient is or comprises allantoin. In some embodiments, the emollient is or comprises panthenol and allantoin.

In some embodiments, hyaluronic acid/hyaluronate acts as gelator and/or emollient.

In some embodiments, the skin moisturizer is provided in a concentration of 0.1-5% (w/w), 0.15-3.0% (w/w), 0.2-1.5% (w/w), 0.4-0.6% (w/w), or around 0.5% (w/w). In some embodiments, the skin moisturizer(s) is or comprises panthenol. In some embodiments, the skin moisturizer(s) is or comprises allantoin. In some embodiments, the skin moisturizer(s) comprises panthenol and allantoin. In some embodiments, panthenol is provided in a concentration of 0.1-5% (w/w), 0.15-3.0% (w/w), 0.2-1.5% (w/w), 0.4-0.6% (w/w), or around 0.5% (w/w). In some embodiments, allantoin is provided in a concentration of 0.01-5% (w/w), 0.05-2% (w/w), 0.1-1% (w/w), 0.2-0.5% (w/w), or around 0.3% (w/w). In some embodiments, panthenol and allantoin is provided in a concentration of 0.01-5% (w/w), 0.05-2% (w/w), 0.1-1% (w/w), 0.2-0.5% (w/w), or around 0.3% (w/w) each. In some embodiments, panthenol and allantoin are provided in a combined concentration of 0.01-5% (w/w), 0.05-2% (w/w), 0.1-1% (w/w), 0.2-0.5% (w/w), or around 0.3% (w/w) each. In some embodiments, the emollient is selected from one or more of: Panthenol, Allantoin, Isopropyl Myristate, pantothenic acid, Sodium Hyaluronate, Squalene, Phenoxyethanol, methyl-paraben, propyl-paraben, ethyl-paraben, butyl-paraben, lanolin, sorbitol, petrolatum, Stearic acid, Shea butter, Glyceryl stearate, Elastin, Hyaluronic acid, Olive oil, Glycerine Pharma 99.5% vegetable gum, rhizobian, and/or Sea Water.

In some embodiments, the composition comprises a skin conditioner. In the context of the present invention, the term “skin conditioner” or “skin essence” is meant to comprise a component or composition providing softening of the skin. Often, a skin conditioner will also hydrate the skin, such as a moisturizer. In some embodiments, the skin conditioner is provided in a concentration of 0.1-5% (w/w), 0.15-3.0% (w/w), 0.2-1.5% (w/w), 0.6-1.0% (w/w), or around 0.8% (w/w). In some embodiments, the skin conditioner(s) is or comprises panthenol. In some embodiments, the skin conditioner(s) is or comprises allantoin. In some embodiments, the skin conditioner(s) comprises panthenol and allantoin. In some embodiments, allantoin is provided in a concentration of 0.1-5% (w/w), 0.12-3.0% (w/w), 0.2-1.5% (w/w), 0.2-0.4% (w/w), or around 0.3% (w/w). In some embodiments, panthenol is provided in a concentration of 0.1-5% (w/w), 0.15-3.0% (w/w), 0.2-1.5% (w/w), 0.4-0.6% (w/w), or around 0.5% (w/w). In some embodiments, allantoin and panthenol are provided in a combined concentration of 0.1-5% (w/w), 0.15-3.0% (w/w), 0.2-1.5% (w/w), 0.6-1.0% (w/w), or around 0.8% (w/w).

In some embodiments, the skin conditioner is selected from one or more of: Panthenol, Astrocaryum Vulgare Seed Butter, Gossypium Hirsutum Seed Extract, Pentaclethra Macrophylla Seed Oil, Abies Alba Extract, Zanthoxylum Bungeanum Pericarp Extract, Zea Mays Germ Extract, Zymomonas Ferment Filtrate, Zingiber Officinale Root, Ziyu Glycoside II, Zostera Marina Callus Extract, Ulva Australis Extract, Actinidia Arguta Juice, Adenosine, Adonis Amurensis Extract, Aloe Barbadensis Leaf Extract, Amaran-thus Spinosus Seed Oil, Ananas Sativus Fruit Juice, Black Soldier Fly Larva Oil, Azur-ite, Bacillus/Corchorus Olitorius Leaf Ferment Filtrate, Cajanus Cajan Leaf Extract, and/or Calcium Polyglutamate Crosspolymer.

In some embodiments, the skin conditioner may provide a further effect, such as a moisturizing effect.

In some embodiments, a skin conditioner may also act as an emollient, and vice versa. An example thereof is e.g. panthenol, which may act as skin conditioner and/or as emollient.

A body cavity composition may benefit from the presence of one or more further wound healing compound(s). In some embodiments, the composition comprises a wound healing compound. In the context of the present invention, a “wound healing compound” is a component that promotes wound healing and/or tissue regeneration. CBD is a further example of a wound healing compound. In some embodiments, the further wound healing compound is, or comprises hyaluronic acid and/or its salt. Suitable concentrations for wound healing compound(s) may vary, such as around 0.1-5% (w/w). In some embodiments, the one or more further wound healing compound(s) is provided in a concentration of 0.1-5% (w/w), 0.2-3% (w/w), 0.3-1% (w/w), 0.35-0.75% (w/w), 0.4-0.6% (w/w), or around 0.5% (w/w). In some embodiments, the further wound healing compound is selected from one or more of: Honey (medical), hyaluronic acid/salt, vit-amin E, Aloe vera, Benzalkonium Chloride 0.13%, Propylene Glycol, Glycerin 20.0%, propolis, Petroleum jelly, curcumin, garlic, carbonoid oil, collagen, sorbitol, silver, Anethum graveolens, Anethum graveolens, Cinnamomum verum, Eucalyptus, Securigera securidaca, Trigonella foenum-graecum, Nelumbo nucifera, Neem leaf extracts, Cham-omilla recutita, nitrofurazone, Bael, Moltkia coerulea, and Allium sativum L. (Amaryl-lidaceae) including any combinations thereof.

In some embodiments, hyaluronic acid/hyaluronate acts as wound healing compound. In some embodiments, hyaluronic acid/hyaluronate acts as one or more of: gelator, emollient, and/or wound healing compound, including any combination(s) thereof.

The CBD-comprising compositions may further comprise a “preservative”. A preservative provides stability and/or increased stability of the composition, such as by preventing the growth of microbial organisms in the compositions, also called “anti-microbial agent” herein. In some embodiments, one or more suitable preservative(s) and/or anti-microbial agent(s) may e.g. be selected from: biocide, methylparabens, ethylpara-bens, propylparabens, butylparabens, organic acid, citric acid, sorbic acid, acetic acid, propionic acid, sulfites, nitrites, sodium sorbate, potassium sorbate, calcium sorbate, benzoic acid, sodium benzonate, potassium benzonate, calcium benzonate, sodium metabisulfite, piropylene glycol, benzaldehyde, butylated hydroxytoluene, butylated hydroxyanisole, formaldehyde donors, botanical extracts, monoglyceride, phenol, mer-cury components and any combination thereof. In some embodiments, one or more suitable anti-microbial agent(s) may be selected from one or more of: organic acid, salt of an organic acid, including any combination thereof. Without wanting to be bound by any theory, it is believed that CBD possesses an anti-microbial effect, probably comparable to some conventional antibiotics. CBD is believed to be active against pathogens, such as Staphylococcus aureus, Streptococcus pneumonie and/or Clostridioides dif-ficile.

A body cavity composition may also benefit from the presence of one or more anti-microbial agent(s) or stabilizers, such as for storability and/or microbial safety of the product. In some embodiments, the anti-microbial agent is or comprises benzalkonium chloride. In some embodiments, the one or more anti-microbial agent(s) is provided in a concentration of 0.01-5% (w/w), 0.02-2% (w/w), 0.04-1% (w/w), 0.075-0.2% (w/w), or around 0.1% (w/w). In some embodiments, the anti-microbial agent is/are provided in a concentration of 0.01-2.5% (w/w), 0.025-0.1.0% (w/w), 0.05-0.5% (w/w), 0.075-0.2% (w/w), or around 0.1% (w/w). In some embodiments, the preservative may provide a further effect, such as pH-adjusting and/or buffering effect. In some embodiments, the anti-microbial agent(s) is/are selected from one or more anti-microbial agents/preservative(s) disclosed herein. For e.g. nasal applications or other applications comprising delicate and/or sensitive mucous membranes, it can be advantageous to use an anti-microbial agent that does not provide discomfort, e.g. a burning and/or stinging sensation when applied in a nostril. In some embodiments, the body cavity composition, such as an intra-nasal composition comprises an anti-microbial agent certified for use in cosmetica, in particular lip balm, lip gloss, lip stick, or eye make-up. Examples of such anti-microbial agents may e.g. comprise one or more of: Cocos Nucifera (Coconut) Fruit Juice (and) Lactobacillus Ferment; Caprylyl Glyceryl Ether; Ethylhexylglycerin; Hexylglycerin; Caprylyl Glycol; Camellia Sinensis Leaf Extract (and) Zingiber Officinale (Ginger) Water (and) Lactobacillus Ferment; Phenylpropanol; Capryloyl Glycine; and or Caprylyl Glycol (and) Glyceryl Caprylate/Caprate (and) Glycerin.

Provision of a defined pH can be achieved using methods known in the field, comprising addition of one or more acid(s), base(s), salt(s) of said acid(s) and/or base(s), buffering agent(s) and/or pH-stabilizer(s), including any combination thereof. In some embodiments, NaOH is used in this context. In some embodiments, other pharmaceutically acceptable acid(s) or base(s) including their salts can be used. In some embodiments, triet

hanolamine and/or citrate/citric acid are used in the provision and/or maintenance of the desired pH. In some embodiments, an alkanolamine such as aminomethyl propanol (AMP) is used as a buffering agent. In some embodiments, a base, such as strong base, in particular NaOH is used to provide the desired pH.

A “chelating agent” or “chelator” is a compound that is capable of forming chelated complexes with ions, such as metal ions. It is usually an organic compound, capable of reacting with a metal ion to produce a chelate. Examples of suitable chelating agents may comprise EDTA, citric acid, tartaric acid, phytic acid, triethanolamine, and salic-ylaldehyde. In some embodiments, phytic acid and/or its salt is used as chelating agent. In some embodiments, the chelator is present in a concentration of 0.075-0.2% (w/w), or around 0.1% (w/w). In some embodiments, the chelator is phytic acid or phytate, such as sodium phytate.

Phytic acid is believed to possess mild keratolytic and exfoliating properties at higher concentration. Phytic acid can inhibit melanin formation by blocking the activity of iron and copper in melanogenesis. Phytic acid possesses chelating properties and can e.g. improves foam quality by reducing the hardness of water. It can be used as an alternative for EDTA and it is compatible with, and it may even be acting synergistically in combination with cosmetic antioxidants. Phytic acid is commonly used in skin care and face-, body-, sun-, hand-, foot-, hair-, eye- and lip care products. It is also suitable for wet wipes, deodorants and shower products. In some embodiments phytic acid is provided as Dermofeel® PA by Dr. Straetmans (Evonik).

Generally, the presence of oil(s) and/or fat(s) is not desired in CBD-comprising compositions according to the invention, in particular in the context of a topical composition formulated as a gel to be applied onto the skin of a subject. In some embodiments, the composition is not formulated as an oil-in-water emulsion or water-in-oil emulsion. Thus, in some embodiments, the composition comprises no, or insignificant amounts, e.g. than 1.0, 0.5 or 0.1% (w/w) oil, such as edible oil, dehydrated oil, and/or dehydrated edible oil. This may seem counter intuitive, as oils/fats are commonly used to keep the skin soft and smooth, in particular in conventional topical compositions, such as wound treatment recipes, such as for skin burns, where e.g. goat fat is used for treatment of burn wounds. However, in the present invention it is believed the potential downs side of a body cavity composition formulated without fat(s)/oil(s) is more than outweighed by the current CBD-comprising recipes, in particular when comprising e.g. hyaluronic acid/hyaluronate. Without wanting to be bound by any theory, it is believed that the presence of such fat(s) and/or oil(s) contributes negatively with respect to the efficacy of the formulation, as CBD is hydrophobic, and oil(s)/fat(s) will form a kind of barrier and/or layer on the skin, thereby impeding relevant active compounds from being able to actively participating in the desired effect. Furthermore, oil(s) and/or fat(s) may hin-der, obstruct, or even destroy the gel, whereby the composition will no longer be able to form a protective layer covering the mucous membrane, such as the nasal mucous membrane. Furthermore, a satisfactory environment and/or effect may no longer be achievable, such as not maintaining a protective film in the (nasal) mucous membrane against undesired compounds/particles, such as dust, pollen, environmental pollution, bacteria and vira.

Consequently, in some embodiments, a CBD-comprising composition as disclosed herein comprises no or only minute amounts of oil(s) and/or fat(s). In some embodiments, the composition comprises less than 1.0, 0.5, 0.1% oil(s) and/or fat(s). In some embodiments, the composition does not comprise one or more of: (i) oil, such as edible oil, (ii) fat, such as edible fat. Generally, compositions disclosed herein do not comprise an oil-in-water or water-in-oil emulsion.

However, the presence of one or more emulsifier(s) can be advantageous in some embodiments, such as when the composition comprises glycols, such as glycols disclosed herein. In some embodiments, the composition comprises an emulsifier, such as an ionic- or non-ionic emulsifier. In some embodiments, the emulsifier is or comprises one or more of lauryl glucoside, polyglyceryl-2 dipolyhydroxystearate.and glycerin, including any combinations thereof. In some embodiments, the emulsifier is EUMULGIN® VL 75 provided by BASF, which is a non-ionic, O/W emulsifier. It is e.g. used in some skin care emulsion especially sprays and lotions.

In some embodiments, a body cavity composition, such as an intranasal composition is e.g. formulated for topical application inside the nostril(s), comprising 0.1-5%, 0.11-2%, 0.12-1%, 0.15-0.25%, or around 0.2% (w/w) CBD; 25-85%, 35-75%, 45-70%, 55-65%, or around 53-54% (w/w) water; and one or more penetrator(s) and/or penetration enhancer(s) such as propylene glycol and/or pentylene glycol; (i) one or more gela-tor(s), such as polyacrylic acid/polyacrylate (e.g. 2-propeonic acid homopolymer and/or Carbomer); and/or hydroxyethyl cellulose, and optionally (ii) one or more skin moist-urizers and/or skin conditioners, such as panthenol and/or allantoin; (iii) one or more further wound healing compound(s), such as hyaluronic acid and/or its salt; (iv) one or more anti-microbial agent(s), such as benzalkonium chloride; (v) one or more pH sta-bilizer(s) and/or buffering agent(s); such as NaOH and/or (vi) one or more chelating agent(s), such as phytic acid and/or its salt; including any combination(s) of (ii)-(vi).

In some embodiments, the body cavity composition comprises components (i) and (ii), and optionally one or more of components (iii)-(vi). In some embodiments, the composition comprises components (i) and (iii), and optionally one or more of components (ii), (iv)-(vi). In some embodiments, the composition comprises components (i) and (iv), and optionally one or more of components (ii), (iii), (v), (vi). In some embodiments, the composition comprises components (i) and (v), and optionally one or more of components (ii)-(iv), and (vi). In some embodiments, the composition comprises components (i) and (vi), and optionally one or more of components (ii)-(v). Components (i)-(vi) are disclosed in detail herein. Furthermore, in some embodiments, apart from any of the above combinations of components (i)-(vi), the composition comprises NaCl, usually in a physiological concentration, such as 0.5-1.5, 0.75-1.05, or around 0.9% NaCl by weight.

Generally, in the context of the present invention, the presence of alcohol, in particular low-molecular weight alcohols, such as C₁-C₄ alcohols is not desired. C₁-C₄ alcohols can e.g. be selected from one or more of methanol, ethanol, propanol, butanol, including any isomers and/or any combination(s) thereof. Commonly, alcohol is used to clean and/or disinfect a wound. However, low-molecular weight alcohol may actually harm sensitive tissue, such as in a body cavity, such as a mucous membrane. Furthermore, applying low-molecular weight alcohol on a sensitive tissue, such as mucous membrane inside of a body cavity, such as a nostril will give an unpleasant feeling of stinging and/or burning. Low-molecular weight alcohol has also a dehydrating effect on the skin, which is undesirable. In some embodiments, the composition comprises no low-molecular weight alcohol, or only small amounts of low-molecular weight alcohol, in particular low molecular weight alcohol, such as one or more C1-C4 alcohol. In some embodiments, the body cavity composition comprises no and/or 0.25% (w/w) or less, such as 0.20% (w/w) or less, or 0.10 (w/w) or less alcohol C1-C4 alcohol.

For intra-body cavity applications, presence of salt, in particular NaCl can be desirable. In some embodiments, the composition comprises NaCl in physiological amounts. Thus in some embodiments, the composition comprises by weight 0.5-2.5% NaCl. In some embodiments, the composition, such as a nose gel comprises 0.5-2.5, 0.7-1.5, 0.8-1.0, or around 0.9% NaCl by weight.

CBD compositions, depending on the production method used, including the variety of Cannabis used. When extracted, they may comprise varying amounts of impurities, such as further cannabinoids. Generally, the presence of such impurities is not desired, especially, when the nature, concentration and/or composition of these impurities is un-known, and/or when they vary significantly from batch to batch. Thus, in some embodiments, the CBD has a purity of at least 95% (w/w), 98% (w/w), 99% (w/w), 99.5 (w/w), or more than 99.8% (w/w). In some embodiments, the CBD is provided as a powder, and/or in “crystalline form”. Generally, without wanting to be bound by any theory, the use of CBD dissolved considered less ideal, such as for the reasons disclosed herein.

However, in some embodiments, a body cavity composition may comprise one or more further cannabinoid(s), such as one or more psychoactive or one or more non-psychoactive cannabinoid(s), e.g. one or more of THC (tetrahydrocannabinol), THCA (tetrahydrocannabinolic acid), CBDA (cannabidiolic acid), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (canna-bichromevarin), CBGV (cannabigerovarin), CBGM (cannabigerol monomethyl ether), CBE (cannabielsoin), and or CBT (cannabicitran), including any combination(s) thereof.

However, most often, significant amounts, in particular physiologically active amounts of one or more further cannabinoids are generally undesirable. Consequently, in some embodiments, the composition does not comprise, or comprises less than 1.5, 1.0, 0.5 or 0.1% (w/w) of one or more further cannabinoid(s), such as one or more psychoactive or one or more non-psychoactive cannabinoid(s), e.g. one or more of THC, THCA, CBDA, CBN, CBG, CBC, CBL, CBV, THCV, THCP, CBDV, CBCV, CBGV, CBGM, CBE, and or CBT, including any combination(s) thereof. In some embodiments, the CBD or composition comprises less than 0.1 (w/w) THC. In some embodiments, the CBD or composition comprises less than 1.5% (w/w) of any one of CBDV, CBDA, CBG, CBN. In some embodiments, the CBD comprises less than 1.0, 0.5, 0.2 or 0.1% (w/w) by weight CBDV, CBDA, CBG, CBN, or THC.

The absence of significant amounts of any further cannabinoids appears counter intuitive and contrary to common belief claiming a positive, synergistic effect of further cannabinoids in e.g. compositions for wound- and/or pain treatment.

Surprisingly and unexpectedly, the inventor has realised that body cavity compositions according to the present invention, such as intra-nasal compositions, optionally formulated as a gel can provide one or more of the following effects:
prevention of allergy by particles, such as pollen, dust, hair and the like;
mitigation and/or reducing the severity of allergy, such as by pollen, dust, hair and the like;
prevention of infection by pathogens, such as vira, microorganisms, bacteria, yeasts or fungi;
mitigation of risk of infection by airborne pathogens, such as vira, microorganisms, bacteria, yeasts or fungi; and/or
Reducing severity of an infection by pathogens, such as vira, microorganisms, bacteria, yeasts or fungi.

Without wanting to be bound by any theory, it is believed that the use of a composition according to the present invention helps to prevent the ingress of infectious agents such as dust, pollen, environmental pollution, bacteria and/or vira, including any combinations thereof. Furthermore, without wanting to be bound by any theory, it is believed that by using the body cavity composition, undesired particles and/or agents are trapped at least in part by the body cavity composition, before the reach further and/or deeper into the body cavity. With respect to an intranasal composition, such particles/agents are trapped before the enter deeper into the respiratory system, such as deeper into the nostrils, and/or even lungs. Apart, from a “mechanical effect” by a composition, which may, so to say, resemble nasal mucous, e.g. snot, compositions according to the present invention may further provide an anti-microbiale effect, and/or anti-viral effect. This anti-microbial and/or anti-viral effect is believed to be provided by one or more of the components of the intra-body cabity composition. This can either be a direct action of the one- or more components against the microorganism and/or virus, or indirectly, by providing a positive effect on the mucous-membrane system, such as a stimulating and/or activating effect. Such positive effects are provided without triggering one or more undesired effects, such as irritation, stinging, burning sensation or the like.

It is believed that body cavity compositions, such as intranasal compositions as present herein: (x) stabilize the nasal mucous membrane; (y) moisturize the (nasal) mucous membrane; and/or (z) due to the appropriate viscosity, are able to maintain a protective film for longer, including any combination(s) of thereof.

In some embodiments, the allergy is a respiratory allergy caused by e.g. pollen, dust, dust mites, hair, skin and/or other airborne, usually protein-comprising particles.

Common signs and symptoms of such respiratory allergies may comprise:
Nose: swelling of the nasal mucosa (allergic rhinitis) runny nose, sneezing;
Sinuses: allergic sinusitis;
Eyes: redness and itching of the conjunctiva (allergic conjunctivitis, watery eyes);
Airways: sneezing, coughing, bronchoconstriction, wheezing and dyspnea, sometimes outright attacks of asthma, in severe cases the airway constricts due to swelling known as laryngeal oedema;
Ears: Feeling of fullness, possibly pain, and impaired hearing due to the lack of eusta-chian tube drainage.

Generally, respiratory allergies are caused by proteins in the air that are inhaled and trigger airway inflammation. They may be due to specific allergic reactions, or more general reactions to irritants such as smoke and fumes in the indoor and outdoor environment that can aggravate allergy symptoms.

In some embodiments, the infection is an STD (sexually transmitted disease).

In some embodiments, the infection is caused by an airborne virus, or microorganism, such as a respiratory virus such as common cold, influenza, including COVID-19.

The respiratory vira that most commonly circulate in all continents as endemic or epidemic agents are believed to be influenza virus/vira (=virus/a), respiratorysyncytial virus, parainfluenza virus/a, metapneumovirus, rhinovirus/a, coronavirus/aa, adenovi-rus/aa, and bocavirus/a.

In some embodiments, the body cavity composition, such as an intranasal composition provides protection and/or defence against pathogens and/or to help prevent the ingress of infectious and/or antigenic agents such as dust, and/or pollen.

In some embodiments the pollen is e.g. selected from one or more of: ragweed, mountain cedar, ryegrass, pigweed/tumbleweed, Arizona cypress, alder, ash, beech, birch, box elder, cedar, cottonwood, date palm, elm, mulberry, hickory, juniper, oak, pecan, Phoenix palm, red maple, silver maple, sycamore, walnut, willow, Bermuda grass, Johnson grass, Kentucky bluegrass, orchard grass, rye grass, sweet vernal grass, Timothy grass, English plantain, lamb's quarters, redroot pigweed, sagebrush, tumbleweed (Russian thistle), Begonia, cactus, chenille, clematis, columbine, crocus, daffodil, dusty miller, geranium, hosta, impatiens, iris, lily, pansy, periwinkle, petunia, phlox, rose, salvia, snapdragon, thrift, tulip, verbena, and/or zinnia.

In some embodiments, the undesired agent or particle is selected from e.g.: environmental pollution, particulate matter (PM), lead, complex organic chemicals, sulphates, nitrates, mineral dust, and water suspended in the air, indoor air pollutants, and/or tobacco particles.

In some embodiments, the undesired bacteria and/or vira are e.g. selected from: Bocavirus, Sinusitis, Pharyngitis, Epiglottitis, common cold, human coronavirus; SARS, MERS, COVID-19 (SARS-COV-2), bronchitis, Bronchiolitis, Croup, avian influenza, swine influenza, adenovirus, enterovirus, human metapneumovirus, rhinovirus (RV), influenza A&B, parainfluenza, respiratory viral infections and respiratory syncytial virus (RSV).

In some embodiments, the present invention concerns a composition suitable for and/or formulated for application in a body cavity, said composition comprising cannabidiol (CBD), glycol(s), non-ionic emulsifier(s), NaCl, panthenol, hyaluronic acid/salt, phytic acid and water. In some embodiments, the composition may further comprise one or more of: gelling agent, allantoin, and/or pH regulator.

In some embodiments, such a composition may comprise one or more components in the following concentrations (by weight):
a) Cannabidiol (CBD): 0.05-2.5%
b) Glycol(s): 12-70%,
c) Non-ionic emulsifier *: 0.5-5.0%
d) NaCl: 0.5-2.5%
e) Panthenol: 0.25-2.0%
f) Hyaluronic acid/salt: 0.2-2.5%
g) Phytic acid: 0.05-1.0%
h) Water: up to 100%

In some embodiments, 5, 6 or all 7 components are provided in the above-listed concentrations.

In some embodiments, the composition may further comprise allantoin, such as 0.015-1.5% allantoin by weight.

In some embodiments, the composition is formulated as a gel. In some embodiments, the composition will thus comprise one or more gelling agent(s). A suitable gelling agent is e.g. hydroxyethylcellulose. In some embodiments, a composition formulated as a gel may comprise 0.5-5.0% gelling agent by weight, such as Hydroxyethylcellulose.

Generally, formulations according to the present invention will be formulated with a suitable pH in terms of the body cavity, into which the gel is intended to be used. In order to provide a defined pH, one or more pH regulators can be provided, such as 0.02-1.0% by weight.

Thus, in some embodiments, apart from 5, 6, or 7 of components (a)-(h), a composition according to the present invention may comprise (by weight):
i) Gelling agent, such as Hydroxyethylcellulose: 0.5-5.0%
j) Allantoin: 0.015-1.5%
k) pH regulator: 0.02-1.0%;
including any combination(s) thereof.

In some embodiments, a CBD-comprising composition is formulated such that a defined pH is provided. Generally, a neutral, near neutral, and/or slightly acidic pH, such as a pH mimicking the pH of the skin is often preferred, such as a pH of around 6.0-6.8, or around 6.5, such as 6.5±0.20, 6.25±0.25, or 6.0±0.25. In some embodiments, a CBD-comprising composition can be formulated with a pH of 5-7, 5-6, 5.5-6.5, or around 6. In some embodiment, the pH is around 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7.0. In some embodiments, the composition is formulated with a pH of 5.0-7.0, 5.2-6.0, 5.4-5.6, or around 5.5. In some embodiments, the pH can be around 5.2-5.8, 5.6-5.7 or around 5.5. In some embodiments, the pH is 5.0-5.2, 5.2-5.4, 5.4-5.6, 5.6-5.8, 5.8-6.0, 6.0-6.2. 6.2-6.4, 6.4-6.6, 6.6-6.8, or 6.8-7.0. In some embodiments, a composition according to the present invention, such as a gel, can be formulated with a neutral or slightly acidic pH, such as pH around 5-7, such as 5.5-6.5, and/or around 5.0, 5.25, 5.5, 5.75, 6.0, 6.25, 6.5, 6.75, or 7.0. In some embodiments, the pH is around 5.2-5.8. In some embodiments, the pH is around 5-5.5.0, 5.5-6.0, 6.0-6.5.0, or 6.5-7.0. In some embodiments, the pH mimics the pH of the body cavity, for which the composition is formulated for. In some embodiments, the pH can also be higher than pH 7. In some embodiments, the pH can be lower than 5.0.

In some embodiments, the glycol(s) is/are selected from one or more of propylene glycol, pentylene glycol, and/or butylene glycol, including any combination(s) thereof.

In some embodiments, the glycol(s) comprise by weight: propylene glycol: 10-50%; pentylene glycol: 1-10%, and/or butylene glycol: 1-10%; including any combination(s) thereof.

In some embodiments, the non-ionic emulsifier consists of, consists essentially of, or comprises Lauryl Glucoside, Polyglyceryl-2 Dipolyhydroxystearate, and Glycerin, such as Eumulgin VL 75.

In some embodiments, the composition is formulated as a gel. Usually, such a composition will comprise one or more gelator(s) and/or gelling agent(s).

Composition according to any one of the preceding claims, formulated as a nose gel for intranasal application.

In some embodiments, the body cavity is a nasal cavity, in particular a nostril.

In some embodiments, a composition is provided, wherein one or more components (a)-(h) are provided in a concentration by weight of:
a) 0.05-2.5, 0.1-1.0, 0.15-2.5, or around 0.2% CBD;
b) 12-70, 20-60, 35-45, or around 40% Glycol(s);
c) 0.5-5.0, 1.0-4.0, 1.5-2.5, or around 2% Non-ionic emulsifier;
d) 0.5-2.5, 0.7-1.5, 0.8-1.0, or around 0.9% NaCl;
e) 0.25-2, 0.3-1.0, 0.4-0.6, or around 0.50% Panthenol:
f) 0.2-2.5, 0.3-1.0, 0.4-0.6, or around 0.5% Hyaluronic acid/salt, such as Sodium Hyaluronate;
g) 0.05-1, 0.10-0.5, 0.075-0.125, or around 0.1% Phytic acid:
h) Water: up to 100%, such as 20-70, 30-60, 50-56, or around 53% water.

In some embodiments, a composition is provided, wherein components (a)-(h) are provided in a concentration by weight of:
a) 0.05-2.5, 0.1-1.0, 0.15-2.5, or around 0.2% CBD;
b) 12-70, 20-60, 35-45, or around 40% Glycol(s);
c) 0.5-5.0, 1.0-4.0, 1.5-2.5, or around 2% Non-ionic emulsifier;
d) 0.5-2.5, 0.7-1.5, 0.8-1.0, or around 0.9% NaCl;
e) 0.25-2, 0.3-1.0, 0.4-0.6, or around 0.50% Panthenol:
f) 0.2-2.5, 0.3-1.0, 0.4-0.6, or around 0.5% Hyaluronic acid/salt, such as Sodium
Hyaluronate;
g) 0.05-1, 0.10-0.5, 0.075-0.125, or around 0.1% Phytic acid:
h) Water: up to 100%, such as 20-70, 30-60, 50-56, or around 53% water.
In some embodiments, a composition is provided, wherein one or more components (i)
(k) are provided in a concentration by weight of:
i) 0.5-5.0, 0.75-2.5, 1.5-2.0, or around 1.75% gelling agent, such as Hydroxyethylcellulose;
j) 0.015-1.5, 0.1-1.0, 0.2-0.4, or around 0.3% Allantoin;
k) 0.02-1.0, 0.075-0.5, 0.1-0.3, or around 0.04%; pH regulator, such as 20% (w/w) NaOH.

In some embodiments, (i)-(k) are provided in a concentration by weight of: i) 0.5-5.0, 0.75-2.5, 1.5-2.0, or around 1.75% gelling agent, such as Hydroxyethylcellulose;
j) 0.015-1.5, 0.1-1.0, 0.2-0.4, or around 0.3% Allantoin;
k) 0.02-1.0, 0.075-0.5, 0.1-0.3, or around 0.04%; pH regulator, such as 20% (w/w) NaOH.

In some embodiments, a composition is provided, comprising:
a) 0.05-2.5, 0.1-1.0, 0.15-2.5, or around 0.2% CBD;
b) 12-70, 20-60, 35-45, or around 40% Glycol(s);
c) 0.5-5.0, 1.0-4.0, 1.5-2.5, or around 2% Non-ionic emulsifier;
d) 0.5-2.5, 0.7-1.5, 0.8-1.0, or around 0.9% NaCl;
e) 0.25-2, 0.3-1.0, 0.4-0.6, or around 0.50% Panthenol:
f) 0.2-2.5, 0.3-1.0, 0.4-0.6, or around 0.5% Hyaluronic acid/salt, such as Sodium Hyaluronate;
g) 0.05-1, 0.10-0.5, 0.075-0.125, or around 0.1% Phytic acid;
h) Water: up to 100%, such as 20-70, 30-60, 50-56, or around 53% water;
wherein the one or more glycol(s) are provided in a concentration by weight of:
10-50, 15-40, 25-35, or around 30% Propylene glycol;
1-10, 2-8, 4-6, or around 5.0% Pentylene glycol; and
1-10, 2-8, 4-6, or around 5.0% Butylene glycol;
   and optionally
i) 0.5-5.0, 0.75-2.5, 1.5-2.0, or around 1.75% gelling agent, such as Hydroxyethylcellulose;
j) 0.015-1.5, 0.1-1.0, 0.2-0.4, or around 0.3% Allantoin;
k) 0.02-1.0, 0.075-0.5, 0.1-0.3, or around 0.04%; pH regulator, such as 20% (w/w) NaOH.

In some embodiments, a composition is provided, comprising:
a) 0.15-2.5, or around 0.2% CBD;
b) 35-45, or around 40% Glycol(s);
c) 1.5-2.5, or around 2% Non-ionic emulsifier;
d) 0.8-1.0, or around 0.9% NaCl;
e) 0.4-0.6, or around 0.50% Panthenol:
f) 0.4-0.6, or around 0.5% Hyaluronic acid/salt, such as Sodium Hyaluronate;
g) 0.075-0.125, or around 0.1% Phytic acid;
h) Water: up to 100%, such as 20-70, 30-60, 50-56, or around 53% water;
wherein the one or more glycol(s) are provided in a concentration by weight of:
25-35, or around 30% Propylene glycol;
4-6, or around 5.0% Pentylene glycol; and
4-6, or around 5.0% Butylene glycol;
   and optionally
i) 1.5-2.0, or around 1.75% gelling agent, such as Hydroxyethylcellulose;
j) 0.2-0.4, or around 0.3% Allantoin;
k) 0.075-0.5, 0.1-0.3, or around 0.04%; pH regulator, such as 20% (w/w) NaOH.

In some embodiments, a nasal gel is provided, comprising or consisting essentially of:

| | Range | % (by |
|---|---|---|
| Component | (% w/w) | weight) |
| Water, demin. | 50.83-56.18 | 53.60 |
| Propylene glycol | 28.50-31.50 | 30.00 |
| Pentylene glycol | 4.75-5.25 | 5.00 |
| Butylene glycol | 4.75-5.25 | 5.00 |
| Non-ionic emulsifier* | 1.90-2.10 | 2.00 |
| Cannabidiol (CBD) | 0.19-021 | 0.20 |
| Hydroxyethyl cellulose** | 1.66-1.84 | 1.75 |
| d-Panthenol | 0.63-0.67 | 0.65 |
| Hyaluronic acid, Na salt | 0.49-0.52 | 0.50 |
| Sodium chloride | 0.86-0.95 | 0.90 |
| Allantoin | 0.29-0.31 | 0.30 |
| Phytic acid | 0.09-0.11 | 0.10 |
| pH adjusting agent*** | as needed | as needed |
| Total | | 100 |

*e..g. EmulginVL75;
**e.g. Tylose H300NG4;
***NaOH (20%) for pH 5.2-5.8

In some embodiments the CBD used in the provision of the composition, such as a body cavity composition, e.g. an intra-nasal composition such as a nasal gel, is crystalline, such as “type A CBD” as disclosed herein. In some embodiments, said CBD is provided as- or capable of forming-needle-like crystals.

In some embodiments, a CBD-comprising composition according to the first aspect may comprise a further cannabinoid, such as a one or more cannabinoid(s) selected from: THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCC (tetrahydrocannabiorcol), THCV (tetrahydrocannabivarin), THCP (tetrahydrocannabiphorol), CBDV (cannabidivarin), CBCV (cannabichromevarin), CBGV (cannabigerovarin), CBGM (cannabigerol monomethyl ether), CBE (cannabielsoin), CBT (cannabicitran), including one or more cannabinoids of the following types: CBG-type, CBC-type, “CBD-type other than CBD”, THC-type, CBN-type, CBE-type, iso-THC-type, CBL-type, CBT-type, including any combination(s) thereof. Such further cannabinoid may comprise hallucinogenic and/or non-hallucinogenic cannabinoids. Generally, non-hallucinogenic cannabinoids are preferred in order to avoid undesired side-effects upon use or treatment with composition(s) comprising such compounds, in particular when they are present in physiologically active amounts.

Further suitable concentrations and/or concentration ranges may be disclosed herein.

Concerning the CBD used in the preparation or formulation of a CBD-comprising composition, such as a topical formulation, in some embodiments, the CBD used in the provision of the composition is crystalline.

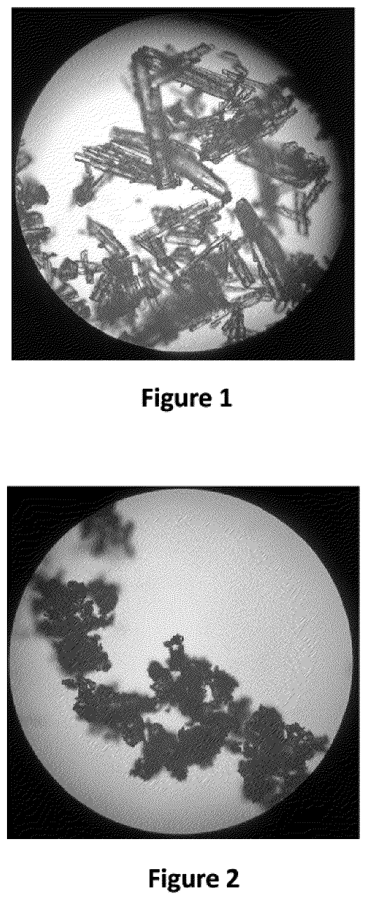

In some embodiments, the CBD used for providing a composition as disclosed above is characterized by one or more features, such as the crystal structure and/or conformation. It has been observed by the inventors, see e.g. Example 7, that CBD with a needle-like crystal structure (=crystal structure A; see FIG. 1), surprisingly and unexpectedly, appears significantly more potent than CBD with a different crystal structure, a non-needle like structure, also termed “bunch-like or “cluster-like” herein (=crystal structure B; see FIG. 2).

In some embodiments, the CBD possesses, when crystalline, or is capable of forming a needle-like crystal structure. In some embodiments, CBD of crystal structure A (or capable of forming needle-like crystals) is at least 1.2, 1.5, 2, 3, 4, 5, 7.5, 10, 15 or 20 times more “potent” or “active” on a weight/weight basis than CBD of crystal structure B (or capable of forming cluster/bunch-like crystals). Often, the “A-type” CBD is at least 2.5, 5, 7.5, or 10 times more “potent” on a weight/weight basis than “B-type” CBD.

The “potency” and/or “activity” of “type A” CBD can e.g. be determined by comparing the effect when using essentially identical compositions with the exception of the type of CBD used. Said potency, activity and or effect can e.g. be determined, mutatis mutandis, as disclosed in the Examples, such as Examples 3-5, by comparing formulations comprising “A type” CBD with formulations comprising “B-type” CBD. Alternatively, the potency can be determined in the quantity of CBD needed to provide the same effect, such as protection against cold or flu, and/or a pathogen. In some embodiments, the use of a more potent CBD results in an increased protection against a pathogen, such as a virus, e.g. cold or flu. In some embodiments, the use of a more potent CBD allows for a reduction of the quantity of CBD used in the formulation of the body cavity composition, in particular a intra-nasal gel composition. In some embodiments, the use of a more potent CBD allows for a reduction of the quantity of formulation necessary to provide a comparable effect, such as a protection against a virus, such as cold, flu and/or COVID 19.

CBD of crystal structure A, or CBD capable of forming needle-like crystals, is also called “type A CBD” herein, while CBD of crystal structure B, or CBD capable of forming “bunch-like or “cluster-like” crystals is called “type B CBD”. In some embodiments, the CBD is “type A CBD”. Often, “type A CBD” is preferred in contrast to “type B CBD”.

It can be speculated, if the CBD needs to be in an active form, such one or more specific conformation(s) in order to be active upon administration to a subject, such as in a topical formulation. Lack of activity or potency can also be caused by a lower uptake rate and/or difficulties in passing through the skin.

Without wanting to be bound by any theory, it is believed that the difference in crystal structure may be caused by a different molecular structure, such as a different conformation. This could e.g. be due to a failure of the subject's body to recognize the “wrong” CBD conformation or the like. It is conceivable that the differences in CBD crystal structure are caused by a different extraction process. In particular, the CBD disclosed in FIG. 1 was provided by an extraction process, comprising extraction with isopropanol, distillation and crystallization with heptane, while the CBD disclosed in FIG. 2 was provided by critical CO2 extraction.

Generally, crystalline CBD can be provided by methods and techniques known in the art, such as by methods disclosed in U.S. Pat. No. 10,413,845 and/or U.S. Pat. No. 10,414,709.

In short, crystalline CBD can be provided from hemp or cannabis (Cannabis sativa) by a method consisting essentially of:
Extracting hemp or cannabis with e.g. isopropanol to produce an extract rich in cannabinoids, THC, CBD and terpenes
Evaporating the solvent portion of the extract to generate a substantially solvent-free extract
Distilling the substantially solvent-free extract to isolate the CBD, and
Crystallizing the distilled, isolated CBD to produce a crystallized, isolated CBD and one or more recrystallization(s) if needed by the use of a suitable organic solvent, such as an alkane, e.g. heptane, commonly followed by
Solvent removal by e.g. vacuum drying. to remove volatile remnants.

Thus, in some embodiments, the CBD crystals used in the formulation of the topical composition, such as the body cavity composition, e.g. intra-nasal composition such as a nasal gel are needle-like crystals, such as crystals shown in FIG. 1. Likewise, in some embodiments, the CBD crystals used in the formulation of the topical composition are not cluster- or bunch-shaped, such as crystals similar to crystals shown in FIG. 2.

In some embodiments, the CBD crystals used in the formulation of the topical composition are not provided by an extraction method comprising critical CO2 extraction.

In some embodiments, the CBD crystals used in the formulation of the topical composition are provided by a method comprising extraction with a C3-C4 alcohol, such as isopropanol, and one or more crystallisations steps with a C6-C8 alkane, such as heptane. In some embodiments, the C3-C4 alcohol is isopropanol. In some embodiments, the C6-C8 alkane is heptane. In some embodiments, the C3-C4 alcohol is isopropanol, and the C6-C8 alkane is heptane. This combination is believed to provide CBD crystals of satisfactory quality, such as absence or reduction in inhibitors and/or the desired conformation of the CBD.

In some embodiments, a suitable CBD product can be obtained when the CBD crystals are provided by a method comprising critical CO2 extraction and one or more crystallisations steps with a C6-C8 alkane, such as heptane.

As seen in Table 1, it can be seen that the Cannabinoid profile of type A and type B CBD can be rather similar.

TABLE 1
Analysis of CBD of crystal structure A versus crystal structure B

| Cannabinoid | | |
|---|---|---|
| profile | Type A | Type B |
| CBD | 99.33% | 98.60% |
| CBDV | 0.39% | 0.19% |
| CBDA | 0.01% | n.d. |
| CBG | n.d. | n.d. |
| CBN | 0.04% | n.d. |
| THC | n.d. | n.d. |

n.d. not detected;
type A CBD was sourced from Enecta, type B CBD was sourced from Pharma Hemp

It is, however, also conceivable that the differences in crystal structure, can relate to and be caused by different extraction processes. Different crystal structures can also be in-dicative of different concentrations of “CBD inhibitors”, and/or different concentrations of “CBD enhancers”. In some embodiments, terpenes, such as naturally occurring terpenes, in particular terpenes found in plants, such as in Cannabis sativa, act as CBD inhibitors, which is not desirable.

Thus, in some embodiments, CBD of crystal structure B alias “type B CBD” can be converted to CBD of crystal structure A alias “type A CBD” (and/or CBD capable of forming crystal structure A) by an organic extraction step and/or recrystallisation step. In such embodiments, it is conceivable that the change in crystal structure is related to the presence of inhibitors that are reduced significantly in the additional extraction and/or crystallization step(s). Alternatively, the organic extraction step may provide a change in conformation of the CBD, rendering it more active again. In some embodiments, recrystallization with heptane can change the B-type CBD into A-type CBD.

In some embodiments, CBD of crystal structure B has been provided by critical CO2 extraction, such as CBD crystals provided by www.pharma-hemp.com and/or following a similar extraction protocol as said manufacturer.

In some embodiments, presence of terpenes and/or terpenoids, in particular Cannabis sativa terpenes or in a CBD-comprising topical composition as disclosed herein, provides one or more undesirable effect(s), such as one or more of: reduced efficiency or potency, inability or reduced ability to recognize the CBD, need for a higher CBD formulation for obtaining similar effect, increase in non-CBD cannabinoids in the formulation. In some embodiments, said composition comprises 0.0001% or less, 0.001% or less, 0.01% or less, or 0.1% or less terpenes, in particular Cannabis sativa terpenes, by weight.

In some embodiments, the crystalline CBD does not comprise significant amounts of terpenes, such as less than 0.1, less than 0.05, less than 0.02, less than 0.01, less than 0.005, less than 0.002, less than 0.001% terpenes by weight.

It is also conceivable that other plant components, such as terpenoids can act as inhibitors. In some embodiments the presence of terpenoids, such as Cannabis sativa terpenoids can be undesirable. In some embodiments, the crystalline CBD does not comprise significant amounts of terpenoids, such as less than 0.1, less than 0.05, less than 0.02, less than 0.01, less than 0.005, less than 0.002, less than 0.001% terpenoids by weight.

In some embodiments, the use of CBD having or capable of providing crystals of crystal structure A, such as shown in FIG. 1 in a CBD-comprising composition as disclosed herein, provides a positive effect, such as one or more of: increased efficiency, possi-bility to reduce total amount of CBD in the formulation, the subject needs less topical composition, such as an intra-nasal formulation to achieve the same effect, improved recognition and/or CBD uptake by the subject's body, reduction in non-CBD canna-binoids in the formulation and/or other impurities.

Generally, compositions according to the first aspect can be provided using methods, procedures and/or unit operations known in the art. In some embodiments, compositions according to the first aspect can be provided as shown herein, such as in the second aspect.

In a second aspect, the present invention pertains to a method for providing a composition according to the first aspect. Generally, methods, devices and/or unit operations known in the field can be used.

In some embodiments, provision of a composition according to the present invention comprises the steps or acts of:
Providing the individual components in suitable amounts
Providing of first composition by mixing water; glycol(s) such as propylene glycol and butylene glycol, an emulgator, such as a non-ionic emulgator, e.g. Eumulgin VL 75; NacCl; panthenol; hyaluronic acid/hyaluronate; and optionally allantoin, such as by stirring, until completely dissolved;
Adding Phytic acid;
Adjusting pH (optional), such as by addition of NaOH;
Providing a second composition by dissolving CBD in a further glycol, such as ethylene glycol;
Combining the first and the second composition;
Stirring the combined compositions until homogeneous; and optionally
Dispensing the composition in receptacles, such as receptacles according to the sixth aspect.

Optionally, in particular when formulating a composition as a gel, such a method will comprise the act of:
Adding a gelling agent, such as hydroxyethylcellulose, under mixing, e.g. after combining the first and the second composition.

In some embodiments, the CBD is crystalline CBD. In some embodiments, the CBD is “type A CBD”. Often, the use of “type A CBD” is preferred in contrast to “type B CBD” or other types of CBD.

In a third aspect, the present invention relates to a composition provided by a method according to the second aspect.

In a fourth aspect, the present invention concerns a composition according to the first, third or eighth aspect for use as a medicament and/or in the treatment, prophylactic treatment, mitigation and/or alleviation of one or more symptom(s) and/or condition(s) related to: allergy by particles, such as pollen, dust, hair and the like; infection by pathogens, such as vira, microorganisms, bacteria, yeasts or fungi; and/or infection by airborne pathogens, such as vira, microorganisms, bacteria, yeasts or fungi.

In some embodiment, the infection(s) is/are STDs (sexually transmitted disease).

In some embodiments, the infection is caused by an airborne virus, or microorganism, such as a respiratory virus such as common cold, influenza, including COVID-19.

The respiratory vira that most commonly circulate in all continents as endemic or epidemic agents are believed to be influenza virus, respiratorysyncytial virus, parainfluenza viruses, metapneumovirus, rhinovirus, coronavira, adenovira, and bocavira.

In some embodiments, the body cavity composition, such as an intranasal composition provides protection and/or defence against pathogens and/or to help prevent the ingress of infectious and/or antigenic agents such as dust, and/or pollen.

In some embodiments the pollen is e.g. selected from one or more of: ragweed, mountain cedar, ryegrass, pigweed/tumbleweed, Arizona cypress, alder, ash, beech, birch, box elder, cedar, cottonwood, date palm, elm, mulberry, hickory, juniper, oak, pecan, Phoenix palm, red maple, silver maple, sycamore, walnut, willow, Bermuda grass, Johnson grass, Kentucky bluegrass, orchard grass, rye grass, sweet vernal grass, Timothy grass, English plantain, lamb's quarters, redroot pigweed, sagebrush, tumbleweed (Russian thistle), Begonia, cactus, chenille, clematis, columbine, crocus, daffodil, dusty miller, geranium, hosta, impatiens, iris, lily, pansy, periwinkle, petunia, phlox, rose, salvia, snapdragon, thrift, tulip, verbena, and/or zinnia.

In some embodiments, the undesired agent or particle is selected from e.g.: environmental pollution, particulate matter (PM), carbon monoxide, lead, complex organic chemicals, sulphates, sulfur dioxide, nitrates, nitrogen dioxide, ground-level ozone, mineral dust, and water suspended in the air, sulfur dioxide, nitrogen dioxide, indoor air pollutants, and/or tobacco particles.

In some embodiments, the undesired bacteria and/or vira are e.g. selected from: Bocavirus, Sinusitis, Pharyngitis, Epiglottitis, common cold, human coronavirus; SARS, MERS, COVID-19 (SARS-COV-2), bronchitis, Bronchiolitis, Croup, avian influenza, swine influenza, adenovirus, enterovirus, human metapneumovirus, rhinovirus (RV), influenza A&B, parainfluenza, respiratory viral infections and respiratory syncytial virus (RSV).

In some embodiments, said use, treatment or alleviation of symptoms comprises application of suitable amount, such as “one drop”, such as 0.01-1.0, 0.015-0.1, 0.025-0.075, or around 0.05 g of the composition in the nasal cavity. In some embodiments, the volume of one drop is around 10-250, 15-100, 25-75, or ˜50 μl.

In some embodiments, said use, treatment or alleviation of symptoms comprises application of the composition 1 or more times per day, such as in intervals of around 6 h.

In some embodiments, an appropriate amount of the composition, such as one drop is distributed onto the inner wall of the outermost part of nostril, such as within the last 2, 1.5, 1.0 or 0.5 cm of the nostril towards the exterior opening.

In a fifth aspect, the present invention concerns a method of treatment, prophylactic treatment, prevention, mitigation and/or alleviation of one or more symptom(s) and/or condition(s) comprising the use of a composition according to the first, third, fourth or eighth aspect, wherein the one or more symptom(s) and/or condition(s) is/are: allergy by particles, such as pollen, dust, hair and the like; infection by pathogens, such as vira, microorganisms, bacteria, yeasts or fungi; and/or infection by airborne pathogens, such as vira, microorganisms, bacteria, yeasts or fungi.

In some embodiment, the infection(s) is/are STDs (sexually transmitted disease).

In some embodiments, the infection is caused by an airborne virus, or microorganism, such as a respiratory virus such as common cold, influenza, including COVID-19.

The respiratory viruses that most commonly circulate in all continents as endemic or epidemic agents are believed to be influenza virus, respiratorysyncytial virus, parainfluenza vira, metapneumovirus, rhinovirus, coronavira, adenovira, and bocava.

In some embodiments, the body cavity composition, such as an intranasal composition provides protection and/or defence against pathogens and/or to help prevent the ingress of infectious and/or antigenic agents such as dust, and/or pollen.

In some embodiments the pollen is e.g. selected from one or more of: ragweed, mountain cedar, ryegrass, pigweed/tumbleweed, Arizona cypress, alder, ash, beech, birch, box elder, cedar, cottonwood, date palm, elm, mulberry, hickory, juniper, oak, pecan, Phoenix palm, red maple, silver maple, sycamore, walnut, willow, Bermuda grass, Johnson grass, Kentucky bluegrass, orchard grass, rye grass, sweet vernal grass, Timothy grass, English plantain, lamb's quarters, redroot pigweed, sagebrush, tumbleweed (Russian thistle), Begonia, cactus, chenille, clematis, columbine, crocus, daffodil, dusty miller, geranium, hosta, impatiens, iris, lily, pansy, periwinkle, petunia, phlox, rose, salvia, snapdragon, thrift, tulip, verbena, and/or zinnia.

In some embodiments, the undesired agent or particle is selected from e.g.: the undesired agent is selected from e.g.: environmental pollution, particulate matter (PM), lead, complex organic chemicals, sulphates, nitrates, mineral dust, and water suspended in the air (e.g. aerosols), indoor air pollutants, and/or tobacco particles.

In some embodiments, the undesired bacteria and/or vira are e.g. selected from: Bocavirus, Sinusitis, Pharyngitis, Epiglottitis, common cold, human coronavirus; SARS, MERS, COVID-19 (SARS-COV-2), bronchitis, Bronchiolitis, Croup, avian influenza, swine influenza, adenovirus, enterovirus, human metapneumovirus, rhinovirus (RV), influenza A&B, parainfluenza, respiratory viral infections and respiratory syncytial virus (RSV).

In some embodiments, said use, treatment, prophylactic treatment, prevention, mitigation and/or alleviation of symptoms, contamination and/or infection comprises application of suitable amount, such as “one drop”, such as 0.01-1.0, 0.015-0.1, 0.025-0.075, or around 0.05 mg of the composition in body cavity, such as the nasal cavity. In some embodiments, the volume of one drop is around 10-250, 15-100, 25-75, or ˜50 μl.

In some embodiments, said use, treatment or alleviation of symptoms comprises application of the composition 1 or more times per day, such as in intervals of around 6 h. In some embodiments, an appropriate amount of the composition, such as one drop is distributed onto the inner wall of the outermost part of nostril, such as within the outermost 0.5-2, or 1-2 cm. Usually, this distance corresponds to the distance of the external opening of the nostril to the beginning of the bony part of the nasal septum and/or nose bone.

In a sixth aspect, the present invention relates to a receptacle comprising a composition according to first, third or fourth aspect.

In some embodiments, the receptacle may comprise use of an applicator adapted to be inserted at least in part into a nostril of a subject. In some embodiments, application of the composition, such as a nose gel for intranasal application comprises the acts of: inserting the applicator slightly (e.g. 0.5-2 cm) into a nostril; dispensing a suitable amount, such as a drop; and turning the applicator such as to distribute the composition onto the inner wall of the outermost part of the nostril.

In some embodiments, an appropriate amount of the composition is dispensed on a fingertip, which is thereafter inserted slightly (e.g. 0.5-2 cm) into the nostril and turned, such as to distribute the composition onto the inner wall of the outermost part of nostril.

In some embodiments, an appropriate amount of the composition is dispensed on a cotton swab, which is thereafter inserted slightly (e.g. 0.5-2 cm) into the nostril and turned, such as to distribute the composition onto the inner wall of the outermost part of nostril.

In some embodiments, the receptacle is adapted to dispense one or more droplets, such as to provide the appropriate dosage in terms of volume and/or weight of the composition for the intended application, such as an intra-nasal application. In some embodiments, the droplet volume is around 10-250, 15-100, 25-75, or around 50 μl. In some embodiments, the droplet weight is around 0.01-0.250, 0.015-0.1, 0.025-0.075, 0.40-0.60 or around 0.050 g.

In some embodiments, the receptacle provides protection of UV and/or visible light, such as to protect degradation and/or deterioration of the composition.

In some embodiments, the receptacle is provided with a re-closable opening, such as a screw cap, such as to protect the composition of evaporation, contamination and/or ox-idation.

In a seventh aspect, the present invention concerns a kit comprising a receptacle according to the sixth aspect, and optionally a packaging.

In some embodiments, the kit comprises one or more further screw caps.

In some embodiments, the kit comprises one or more further applicators.

The provision of one or more further applicators and/or screw caps allows for a more hygienic use of the composition. Furthermore, applicators can be provided in different sizes, such as to facilitate use in cases of very different nostril sizes, which can differ significantly between subjects, such as infants to adults.

In an eighth aspect, the present invention concerns a CBD-comprising composition, wherein the CBD used in the formulation is crystalline and/or of “type A”. In some embodiments, said composition is a topical composition as disclosed herein, such as a body cavity composition, such as a nose-gel as disclosed herein, e.g. according to the first, third and/or fourth aspect. In some embodiments, the CBD is of type A (needle-like crystals) or capable of forming needle-like crystals as disclosed herein, e.g. in the first aspect and/or in the Examples.

In a ninth aspect, the present invention pertains to a dosage regimen, comprising administering a topical composition, in particular CBD-comprising topical composition as disclosed herein. In some embodiments, the CBD is of “type A”.

### EXAMPLES

Generally, percentages are % by weight. Crystalline CBD is sourced from Enecta, unless indicated otherwise.

### Example 1

CBD-comprising compositions according to the present invention can be provided using methods, unit operations, protocols and/or know-how customary in the field. This can e.g. be performed as disclosed herein, such as according to the third aspect of the invention, and/or in particular, according to the following Examples.

### Preparation of the Compositions

Step/Phase I:
water
Pentylene glycol
Butylene glycol
Eumulgin VL 75
Sodium chloride
Panthenol
Hyaluronic acid
Allantoin
Mix and stir until it has completely dissolved (heat up)

Step/Phase II:
Add Dermofeel and adjust the pH to approx. PH 5.5
Step/Phase III:
Propylene glycol
CBD
CBD is dissolved inpropylene glycol and stirred.
Phase II and III are combined and stirred until homogeneous

Step/Phase IV:
Tylose H 300 NG4
Disperse and add

| | | |
|---|---|---|
| Component | Range | Quantity |

| | | | | |
|---|---|---|---|---|
| Water, demin. | 50.83-56.18 | kg | 53.50 | kg |
| Propylene glycol | 28.50-31.50 | kg | 30.00 | kg |
| Pentylene glycol | 4.75-5.25 | kg | 5.00 | kg |
| Butylene glycol | 4.75-5.25 | kg | 5.00 | kg |
| EmulginVL75* | 1.90-2.10 | kg | 2.00 | kg |
| Cannabidiol (CBD) | 0.19-021 | kg | 0.20 | kg |
| Tylose H300NG4** | 1.66-1.84 | kg | 1.75 | kg |
| d-Panthenol | 0.63-0.67 | kg | 0.65 | kg |
| Hyaluronic acid, Na salt | 0.49-0.52 | kg | 0.50 | kg |
| Sodium chloride | 0.86-0.95 | kg | 0.90 | kg |
| Allantoin | 0.29-0.31 | kg | 0.30 | kg |
| Dermofeel Phytic acid**** | 0.19-0.21 | kg | 0.20 | kg |

| NaOH (20%) | as needed* | e.g. 0.20 kg | |
|---|---|---|---|
| Total | | 100 | kg |

*EmulginVL75;
**Tylose H300NG4;
*** Dermofeel PA comprises 50% phytic acid;
****e.g. pH 5.2-5.8

### Intra Nasal Composition A

| | | | | % active |
|---|---|---|---|---|
| | | % active | | ingredient |
| | | ingredient | | in final |
| Components | INCI | in component | Amount % | composition |
| Water demin. | Aqua | 100 | 53.5 | 53.5 |
| Propylenglykol USP | Propylene Glycol | 100 | 30 | 30 |
| Hydrolite-5 616751, | Pentylene Glycol | 100 | 5 | 5 |
| Pentylene Glycol 111900, | | | | |
| Microcare Emollient PTG | | | | |
| Butylenglykol | Butylene Glycol | 100 | 5 | 5 |
| Eumulgin VL 75 | Lauryl Glucoside, | 100 | 2 | 2 |
| | Polyglyceryl-2 | | | |
| | Dipolyhydroxystearate, | | | |
| | Glycerin | | | |
| CBD | Cannabidiol | 100 | 0.2 | 0.2 |
| Tylose H 300 NG4 | Hydroxyethylcellulose | 100 | 1.75 | 1.75 |
| Natriumchlorid Ph.Eur. | Sodium Chloride | 100 | 0.9 | 0.9 |
| Panthenol 75% USP | Panthenol | 75 | 0.65 | 0.49 |
| Hyaluronsäure, Na-Salz | Sodium Hyaluronate | 100 | 0.5 | 0.5 |
| 1.0-1.5 Mio. | | | | |
| Allantoin | Allantoin | 100 | 0.3 | 0.3 |
| Dermofeel PA | Phytic Acid | 50 | 0.2 | 0.1 |
| Natriumhydroxid reinst, | Aqua, Sodium | 20 | 0.2 | 0.04 |
| Ph Eur, 20% ig | Hydroxide | | | |
| | | | Sum | |
| | | total | 100 | 99.78 |

| | | | | |
|---|---|---|---|---|
| Water demin. | Aqua | 100 | 97.15 | 97.15 |
| Tylose H 300 NG4 | Hydroxyethylcellulose | 100 | 1.75 | 1.75 |
| Natriumchlorid Ph.Eur. | Sodium Chloride | 100 | 0.9 | 0.9 |
| Natriumhydroxid reinst, | Aqua, Sodium | 20 | 0.2 | 0.04 |
| Ph Eur, 20% ig | Hydroxide | | | |
| | | total | 100 | 99.84 |

### Example 2-Inclusion/Exclusion Criteria

Ages Eligible for Study 18 Years to 65 Years (Adult, Older Adult)
Sexes Eligible for Study: All
Accepts Healthy Volunteers: Yes

INCLUSION CRITERIA:
Age: 18 to 65 years of age
   Healthy participants, as determined by screening assessments and Principal Investigator's judgment
   Healthy status is defined by the absence of evidence of any active or chronic disease following a detailed medical and surgical history, a complete physical examination including vital signs.
   Body Mass Index (BMI) of 18-30 kg/m2 inclusive, with body weight in the range of 50-100 kg

Available to participate for the planned duration of the investigational study for which the screening is being done.

Able and willing to complete the informed consent process.

EXCLUSION CRITERIA:

Any clinically relevant history or the presence of e.g. respiratory, renal, hepatic, gastro-intestinal, hematological, lymphatic, neurological, cardiovascular, psychiatric, etc. disease or diseases-Known to be infected with HIV, syphilis, tuberculosis, hepatitis B or hepatitis C.
Disorders of central nervous system, psychiatric disorders, behavioral disturbances (e.g. cerebrovascular events, depression, post-traumatic stress disorder [PTSD], anxiety, bipolar disorder, severe migraine, Parkinson's disease)·
Participation in an investigational drug or device study within 3 months prior to screening or more than 4 times per year
Concomitant disease or condition that could interfere with, or treatment of which might interfere with, the conduct of the study, or that would, in the opinion of the investigator, pose an unacceptable risk to the subject in this study

A condition that requires active medical intervention or monitoring to avert serious dan-ger to the participant's health or well-being.

Known to be pregnant or breast-feeding.

### Example 3 Test Setup

Test of protective nasal gel alone or in combination of immune booster (food supple-ment) against flue (incubation time: 1-4 days) or cold (incubation time: 1-3 days).

Healthy people exposed to people with the Flu or a Cold.

See Example 2 for inclusion/exclusion criteria.

Ordinal Scale for Clinical Improvement

Recently, a novel ordinal scale end point of hospitalized patient status was introduced in a post hoc analysis of outcomes in a randomized controlled trial (RCT) of immune plasma. This scale placed patients into 1 of 8 mutually exclusive clinical categories, ranging from 1 (discharge from hospital with usual function) to 8 (death).

The primary aim is to prevent test subject to become ill, in particular to keep them out of the ordinal scale for clinical improvement, while exposed to people with the flu or a cold. (a 8-category ordinal scale that ranges from 1 (discharged with normal activity) to 8 (death), or hospital discharge up to day 28).

Temperature measurement by ear: Body temperature may be abnormal due to fever (high temperature) or hypothermia (low temperature). A fever is indicated when body temperature rises about one degree or more over the normal temperature of 370° C.

How to measure temperature-during rest: by ear thermometer

Respiratory rate: In adults, the cut-off for an elevated respiratory rate is usually considered a rate over 20 breaths per minute, with a rate of over 24 breaths per minute indi-cating a very serious condition (when it is related to a physical condition rather than a psychological condition such as a panic attack).

Fever: An increased rate of breathing with a fever is the body's attempt to lose heat by breathing faster. This is important both because a rapid respiratory rate can be a sign of a worsening infection, and because a fever needs to be taken into account in interpreting the respiratory rate.

Infections: Common and uncommon infections such as the cold, flu, pneumonia, and tuberculosis can result in rapid breathing.

How to measure-during rest: To measure the respiratory rate, count the number of breaths for an entire minute

The normal pulse for healthy adults ranges from 60 to 100 beats per minute. The pulse rate may fluctuate and increase with exercise, illness (like flu and Cold, injury, and emotions).

How to measure-during rest:

You can easily check your pulse on the inside of your wrist, below your thumb.
1. Gently place 2 fingers of your other hand on this artery.
2. Do not use your thumb because it has its own pulse that you may feel.
3. Count the beats for 30 seconds; then double the result to get the number of beats per minute or count the beats for 60 seconds.

Another way to tell the difference between the two illnesses is how quickly the symptoms come on. While cold symptoms tend to creep up a little at a time, the flu tends to come on a lot quicker, according to WebMD.

The reason the common cold and the seasonal flu are hard to tell apart might be the fact that they share a lot of similarities: They are both caused by vira, they are both respiratory illnesses and they-along with most other illnesses—can cause your heart to beat a little faster than normal. The heart functions differently when the body is fighting an infection, according to MD Health, but if your resting heart rate ever rises above 85, see your doctor immediately.

As of March 2020, the Center for Disease Control and Prevention (CDC) notes that symptoms including fever, coughing and shortness of breath appearing within two and 14 days of exposure to someone who might have been exposed to the novel coronavirus (COVID-19) may mean it is time to contact your healthcare provider as more news about community spread of the virus develops.

### Example 4-how to Use Nasal Gel

The gel can be applied up to 2 times a day with at least six hours between applications. A suitable quantity, such as a drop is provided by squeezing the container. Said drop is than applied onto the skin/mucous membrane in the nose by introducing the nasal applicator carefully and not too deeply into your nose (e.g. not deeper than the nose bone and/or bony part of the nasal septum) to avoid injuries.

If the amount used is accidentally too high and is perceived as unpleasant, the excess amount can be removed by blowing your nose vigorously into a handkerchief.

### Example 5 Test Results

TABLE A
Protective Intranasal Gel

| | | | | | | | | |
|---|---|---|---|---|---|---|---|---|
| Test person - Gender Male—M or | F-35 | F-43 | F-51 | M-53 | F-55 | M-61 | F-26 | F-39 |
| Female—F & Age | | | | | | | | |
| Exposed to Flue—F or Cold—C | C | C | C | C | C | C | F | F |
| Test period(days) minimum 21 | 21 | 21 | 30 | 30 | 30 | 30 | 21 | 30 |
| days - max 60 days | | | | | | | | |
| Body temperature, 30 min. | B: 36.5 | B: 37 | B: 37 | B: 37 | B: 37 | B: 37 | B: 37 | B: 37 |
| before first dosing on day 1 - | | | | | | | | |
| average or highest in test period | A: 36.5 | High: 37.5 | A: 37 | A: 37 | A: 37 | A: 37 | A: 37 | A: 37 |
| (measured every 24 hours) | | | | | | | | |
| Pulse rate, 30 min. before first | B: 81 | B: 76 | B: 82 | B: 85 | B: 91 | B: 96 | B: 78 | B: 82 |
| dosing on day 1 - average in test | | | | | | | | |
| period (measured every 24 hours) | A: 80 | A: 83 | A: 83 | A: 86 | A: 90 | A: 95 | A: 77 | A: 84 |
| Respiratory rate: Normal | B: 14 | B: 12 | B: 16 | B: 13 | B: 15 | B: 16 | B: 12 | B: 14 |
| respiration rates for an adult person at | | | | | | | | |
| rest range from 12 to 16 breaths | A: 13 | A: 16 | A: 16 | A: 12 | A: 14 | A: 15 | A: 12 | A: 15 |
| per minute. | | | | | | | | |
| Illness signs: coughing, fatigue, | None | Runny nose, | None | None | None | None | None | None |
| and shortness of breath | | sneeze, | | | | | | |
| cough, sneeze, runny nose, fever | | fatigue | | | | | | |
| 7-category ordinal scale, where 0 | 0 | 1 | 0 | 0 | 0 | 0 | 0 | 0 |
| healthy no sign of illness - 8 death | | | | | | | | |
| Medication taken during test | NA | Paracetamol | NA | NA | NA | NA | NA | NA |
| period? | | | | | | | | |

TABLE B
Placebo Intranasal Gel

| | | | | | | | | |
|---|---|---|---|---|---|---|---|---|
| Test person - Gender Male—M or | M-39 | M-45 | F-23 | F-39 | F-36 | F-61 | F-59 | M-60 |
| Female—F & Age | | | | | | | | |
| Exposed to Flue—F or Cold—C | F | C | F | C | C | C | C | C |
| Test period(days) minimum 21 | 21 | 23 | 21 | 30 | 21 | 30 | 30 | 30 |
| days - max 60 days | | | | | | | | |
| Body temperature, 30 min. | B: 37 | B: 37 | B: 37 | B: 37 | B: 36.5 | B: 37 | B: 37 | B: 37 |
| before first dosing on day 1 - | | | | | | | | |
| average or highest in test period | High: 38.5 | High: 37.5 | A: 37 | High: 37.5 | High: 37 | High: 38 | A: 37 | High: 37.5 |
| (measured every 24 hours) | | | | | | | | |
| Pulse rate, 30 min. before first | B: 81 | B: 80 | B: 76 | B: 82 | B: 78 | B: 91 | B: 85 | B: 87 |
| dosing on day 1 - average in test | | | | | | | | |
| period (measured every 24 hours) | A: 93 | A: 86 | A: 77 | A: 84 | A: 84 | A: 97 | A: 87 | A: 92 |
| Respiratory rate: Normal | B: 14 | B: 12 | B: 12 | B: 12 | B: 13 | B: 16 | B: 15 | B: 14 |
| respiration rates for an adult person at | | | | | | | | |
| rest range from 12 to 16 breaths | A: 21 | A: 16 | A: 12 | A: 15 | A: 18 | A: 21 | A: 16 | A: 18 |
| per minute. | | | | | | | | |
| Illness signs: coughing, fatigue, | Fever, cough, | Runny nose, | None | Cough, | Runny nose, | Coughing, | None | Cough, |
| and shortness of breath, sneeze, | runny nose, | sneeze, | | Runny nose, | cough | fatigue, | | Runny nose, |
| runny nose, fever | fatigue | fatigue | | sneeze, | sneeze, | shortness of | | sneeze, |
| | | | | fatigue | fatigue | breath, | | fatigue |
| | | | | | | sneeze, | | |
| | | | | | | runny nose, | | |
| | | | | | | fever | | |
| 7-category ordinal scale, where 0 | 1 | 1 | 0 | 1 | 1 | 1 | 0 | 1 |
| healthy no sign of illness - 8 death | | | | | | | | |
| Medication taken during test | Paracetamol | NA | NA | NA | Paracetamol | Paracetamol | NA | NA |
| period? | | | | | | | | |

### Example 6-Provision of CBD by Alcohol Extraction, Distillation and Crystallization

Crystalline CBD can be provided by methods and techniques known in the art, such as by methods disclosed in U.S. Pat. No. 10,413,845 and/or U.S. Pat. No. 10,414,709.

In short, crystalline CBD can be provided from hemp or cannabis (Cannabis sativa) by a method consisting essentially of:
Extracting hemp or cannabis with a solvent selected from the group consisting of propanol, isopropanol, butanol, pentanol, hexanol, heptanol, and octanol to produce an extract consisting essentially of an extracted hemp or cannabis consisting essentially of tetrahydrocannabinol, a terpene, or cannabidiol;
Evaporating the solvent portion of the extract to generate a substantially solvent-free extract comprising CBD;
Distilling the substantially solvent-free extract to isolate the CBD, and
Crystallizing the distilled, isolated CBD to produce a crystallized, isolated CBD.

Often, the crystallized, isolated CBD is subjected to vacuum drying to remove volatile remnants, in particular the solvent used in crystallizing or re-crystallizing, if needed.

In particular, a method comprising extraction with isopropanol and crystallization by the use of heptane, including one or more optional re-crystallization steps, followed by vacuum drying can provide CBD with crystal structure A, i.e. needle like crystals. Furthermore, such a CBD can be very low in undesired compounds, such as terpenes.

GC chromatography or other analytical methods known in the art can be used to monitor the process such as to ensure a high yield and/or a high purity of the desired product.

Concerning the raw material, hemp comprising e.g. 2-3% CBD is dried and ground before extraction with isopropanol, such as food grade isopropanol.

Guidance for choosing the appropriate reaction based on the boiling points or ranges of the different compounds can e.g. be found here: www.nwsci.com/customer/docs/SKU-Docs/RMR/Technical % 20Data_Extractions_03.28.18.pdf.

CBD with crystal structure A can e.g. be provided from www.enecta.com, and/or following a similar extraction and/or purification protocol as said manufacturer.

### Example 7-Comparison of Compositions Formulated with Different Crystalline CBDs

Two compositions are prepared according to Example 1, e.g. formulation A, the only difference being that the crystalline CBD used in the formulation is either of type A (needle-like crystals; FIG. 1) or type B (bunch/cluster-like; FIG. 2).

Type A crystalline CBD is sourced from Enecta, while type B CBD is sourced from Pharma Hemp.

When testing both compositions, surprisingly and unexpectedly, it is seen and/or it can be concluded that type A CBD is significantly more active in protection against pathogens, such as virus, e.g. cold and/or flu than type B CBD. Such a test can e.g. be performed, mutatis mutandis, according to the Examples, such as Examples 3-5.

1-42. (canceled)

43. A composition suitable for application in a body cavity of a subject, said composition comprising by weight:
a) Cannabidiol (CBD): 0.05-2.5%;
b) glycol(s): 12-70%;
c) an emulsifier: 0.5-5.0%;
d) NaCl: 0.5-2.5%;
e) Panthenol: 0.25-2.0%;
f) hyaluronic acid/salt: 0.2-2.5%;
g) phytic acid: 0.05-1.0%; and
h) water: up to 100%.

44. The composition according to claim 43, formulated as a gel.

45. The composition according to claim 44, further comprising by weight one or more of:
i) a gelling agent: 0.5-5.0%;
j) Allantoin: 0.015-1.5%;
k) a pH regulator: 0.02-1.0%; or
l) any combination(s) of i), j) and k).

46. The composition according to claim 43, wherein the glycol(s) comprise by weight:
propylene glycol: 10-50%;
pentylene glycol: 1-10%;
Butylene glycol: 1-10%; or
any combination(s) thereof.

47. The composition according to claim 43, wherein the emulsifier is a non-ionic emulsifier comprising lauryl glucoside, polyglyceryl-2 dipolyhydroxysterate and glycerin.

48. The composition according to claim 47 wherein the non-ionic emulsifier comprises Eumulgin VL 75.

49. The composition according to claim 43, wherein the body cavity is a nasal cavity and the composition is formulated as a nose gel suitable for inter-nasal application.

50. The composition according to claim 45, wherein one or more components (a)-(k) are provided in a concentration by weight of:
a) 0.05-2.5, 0.1-1.0, 0.15-2.5, or around 0.2% CBD;
b) 12-70, 20-60, 35-45, or around 40% glycol(s);
c) 0.5-5.0, 1.0-4.0, 1.5-2.5, or around 2% Non-ionic emulsifier;
d) 0.5-2.5, 0.7-1.5, 0.8-1.0, or around 0.9% NaCl;
e) 0.25-2, 0.3-1.0, 0.4-0.6, or around 0.50% Panthenol:
f) 0.2-2.5, 0.3-1.0, 0.4-0.6, or around 0.5% hyaluronic acid, hyaluronate or hyaluronic acid, Hyaluronate/salt;
g) 0.05-1, 0.10-0.5, 0.075-0.125, or around 0.1% Phytic acid;
h) Water: up to 100%, such as 20-70, 30-60, 50-56, or around 53% water;
i) 0.5-5.0, 0.75-2.5, 1.5-2.0, or around 1.75% gelling agent;
j) 0.015-1.5, 0.1-1.0, 0.2-0.4, or a round 0.3% Allantoin; and
k) 0.02-1.0, 0.075-0.5, 0.1-0.3, or around 0.04%; pH regulator.

51. The composition according to claim 46, wherein one or more glycol(s) are provided in a concentration by weight of:
10-50, 15-40, 25-35, or around 30% propylene glycol;
1-10, 2-8, 4-6, or around 5.0% pentylene glycol; or
1-10, 2-8, 4-6, or around 5.0% butylene glycol.

52. The composition according to claim 43, wherein the CBD has a purity of at least 95% (w/w), 98% (w/w), 99% (w/w), 99.5 (w/w), or more than 99.8% (w/w).

53. The composition according to claim 52, wherein the CBD used in the provision of the CBD-comprising composition is crystalline; and wherein the CBD crystals are selected from one or more of:
needle-like crystals;
crystals not provided by an extraction method comprising critical CO2 extraction;
crystals provided by a method comprising extraction with a C₃-C₄ alcohol, and one or more crystallisations steps with a C₆-C₈ alcohol; or
any combination thereof.

54. The composition according to claim 53, wherein the C₃-C₄ alcohol is isopropanol, and the C₆-C₈ alkane is heptane.

55. The composition according to claim 43, wherein the CBD does not comprise, or comprises less than 1.5, 1.0, 0.5 or 0.1% (w/w) of one or more further cannabinoid(s) selected from one or more psychoactive or one or more non-psychoactive cannabinoid(s), THC (tetrahydrocannabinol), THCA (tetrahydrocannabinolic acid), CBDA (cannabidiolic acid), CBN (cannabinol), CBG (cannabigerol), CBC (cannabichromene), CBL (cannabicyclol), CBV (cannabivarin), THCV (tetrahydrocannabivarin), THCP (tetrahydrocannabiphorol), CBDV (cannabidivarin), CBCV (cannabichromevarin), (cannabigerovarin), CBGM (cannabigerol monomethyl ether), CBE (cannabielsoin), CBT (cannabicitran), or any combination(s) thereof.

56. A method for preparing the composition of claim 45, said method comprising the steps or acts of:
providing the individual composition components in the amounts identified in claim 45;
providing a first composition by mixing water; glycol(s), Emulgin VL 75; NaCl;
panthenol; hyaluronic acid/hyaluronate; until completely dissolved;
adding Phytic acid to the first composition;
providing a second composition by dissolving CBD in glycol;
combining the first composition and the second composition; and
stirring the combined compositions until homogeneous to provide a third composition.

57. The method according to claim 56, wherein a gelling agent comprising hydroxyethylcellulose is added to the first, second, or third composition under mixing prior to administering the composition to a body cavity of a subject.

58. A method of treating or preventing one or more symptom(s) or condition(s) in a subject having a symptom or condition related to a disease, allergy, pollutant, infection or an undesirable agent as defined in (a), said method comprising:
a) administering to a body cavity of the subject the composition of claim 43, wherein:
the subject has a symptom or condition related to an allergy to pollen, dust or hair, the subject has a symptom or condition related to an infection from a pathogen comprising a virus, a microorganism, a bacteria, yeast or fungi; the subject has a symptom or condition related to a virus and the virus comprises a respiratory virus, said respiratory virus being a common cold virus, an influenza virus, a respiratory syncytial virus, a parainfluenza vira, metapneumovirus, rhinovirus, coronavira, adenovira, bocavira, Sinusitis, Pharyngitis, Epiglottitis, human coronavirus, SARS, MERS, COVID-19 (SARS-COV-2), bronchitis, Bronchiolitis, Croup, avian influenza, swine influenza, adenovirus, enterovirus, human metapneumovirus, rhinovirus (RV), influenza A&B, parainfluenza, or respiratory syncytial virus (RSV),
wherein: the pollen comprises a pollen from: ragweed, mountain cedar, ryegrass, pigweed/tumbleweed, Arizona cypress, alder, ash, beech, birch, box elder, cedar, cottonwood, date palm, elm, mulberry, hickory, juniper, oak, pecan, Phoenix palm, red maple, silver maple, sycamore, walnut, willow, Bermuda grass, Johnson grass, Kentucky bluegrass, orchard grass, rye grass, sweet vernal grass, Timothy grass, English plantain, lamb's quarters, redroot pigweed, sagebrush, tumbleweed (Russian thistle), Begonia, cactus, chenille, clematis, columbine, crocus, daffodil, dusty miller, geranium, hosta, impatiens, iris, lily, pansy, periwinkle, petunia, phlox, rose, salvia, snapdragon, thrift, tulip, verbena, or zinnia;
the undesirable agent comprises an agent capable of causing environmental pollution, PM, lead; or a complex organic chemical, sulphate, nitrate, or mineral dust; and
the undesirable agent is an indoor pollutant or a tobacco particle in water suspended in air, and the infection is a sexually transmitted disease (STD); and
(b) treating or preventing one or more symptom(s) or condition(s) related to the disease, allergy, pollutant, infection or undesirable agent in the subject.

59. The method according to claim 58 wherein the body cavity of the subject is a nasal cavity, and the composition is administered in an amount of 0.01-1.0, 0.015-0.1, 0.025-0.075, or around 0.05 g.

60. The method according to claim 58 wherein the composition is administered 1 or more times per day or about every 6 hours per day.
